SEQUENCE LISTING (86 sequences)

SEQ ID NO: 1 - protein, 14 aa, Artificial
  synthetic peptide
  MOD_RES (1)..(1): 4-fluorobenzoyl-arginine
  MOD_RES (3)..(3): 3-((2-naphthyl) alanine
  MOD_RES (6)..(6): citrulline
  MOD_RES (8)..(8): D-lysine
  MOD_RES (12)..(12): citrulline
  MOD_RES (14)..(14): C′ AMIDATED
XRXCYXKXPYRXCR

SEQ ID NO: 2 - protein, 14 aa, Artificial
  SYNTHETIC PEPTIDE
  MOD_RES (1)..(1): N′ ACETYLATION
  MOD_RES (3)..(3): 3-((2-naphthyl) alanine
  MOD_RES (6)..(6): citrulline
  MOD_RES (8)..(8): D-lysine
  MOD_RES (12)..(12): citrulline
RRXCYXKXPYRXCR

SEQ ID NO: 3 - protein, 14 aa, Artificial
  SYNTHETIC PEPTIDE
  MOD_RES (1)..(1): N′ ACETYLATION
  MOD_RES (3)..(3): 3-((2-naphthyl) alanine
  MOD_RES (8)..(8): D-citrulline
  MOD_RES (12)..(12): citrulline
RRXCYRKXPYRXCR

SEQ ID NO: 4 - protein, 14 aa, Artificial
  synthetic peptide
  MOD_RES (1)..(1): N′ ACETYLATED
  MOD_RES (3)..(3): 3-((2-naphthyl) alanine
  MOD_RES (6)..(6): citrulline
  MOD_RES (8)..(8): D-citrulline
  MOD_RES (12)..(12): citrulline
RRXCYXKXPYRXCR

SEQ ID NO: 5 - protein, 14 aa, Artificial
  synthetic peptide
  MOD_RES (1)..(1): N′ ACETYLATED
  MOD_RES (3)..(3): 3-((2-naphthyl) alanine
  MOD_RES (6)..(6): citrulline
  MOD_RES (8)..(8): D-lysine
  MOD_RES (11)..(12): citrulline
RRXCYXKXPYXXCR

SEQ ID NO: 6 - protein, 14 aa, Artificial
  synthetic peptide
  MOD_RES (1)..(1): N′ acetylated citrulline
  MOD_RES (3)..(3): 3-((2-naphthyl) alanine
  MOD_RES (6)..(6): citrulline
  MOD_RES (8)..(8): D-lysine
  MOD_RES (12)..(12): citrulline
XRXCYXKXPYRXCR

SEQ ID NO: 7 - protein, 14 aa, Artificial
  synthetic peptide
  MOD_RES (1)..(1): N′ acetylated citrulline
  MOD_RES (3)..(3): 3-((2-naphthyl) alanine
  MOD_RES (8)..(8): D-citrulline
  MOD_RES (12)..(12): citrulline
XRXCYRKXPYRXCR

SEQ ID NO: 8 - protein, 14 aa, Artificial
  synthetic peptide
  MOD_RES (1)..(1): N′ ACETYLATED
  MOD_RES (3)..(3): 3-((2-naphthyl) alanine
  MOD_RES (8)..(8): D-citrulline
  MOD_RES (11)..(12): citrulline
RRXCYRKXPYXXCR

SEQ ID NO: 9 - protein, 14 aa, Artificial
  synthetic peptide
  MOD_RES (1)..(1): N′ ACETYLATED citrulline
  MOD_RES (3)..(3): 3-((2-naphthyl) alanine
  MOD_RES (8)..(8): D-lysine
  MOD_RES (11)..(12): citrulline
XRXCYRKXPYXXCR

SEQ ID NO: 10 - protein, 14 aa, Artificial
  synthetic peptide
  MOD_RES (1)..(1): N′ ACETYLATED
  MOD_RES (3)..(3): 3-((2-naphthyl) alanine
  MOD_RES (6)..(6): citrulline
  MOD_RES (8)..(8): D-citrulline
  MOD_RES (12)..(12): citrulline
  MOD_RES (14)..(14): C′ AMIDATED
RRXCYXKXPYRXCR

SEQ ID NO: 11 - protein, 14 aa, Artificial
  synthetic peptide
  MOD_RES (1)..(1): N′ ACETYLATED
  MOD_RES (3)..(3): 3-((2-naphthyl) alanine
  MOD_RES (6)..(6): citrulline
  MOD_RES (8)..(8): D-lysine
  MOD_RES (11)..(12): citrulline
  MOD_RES (14)..(14): C′ AMIDATED
RRXCYXKXPYXXCR

SEQ ID NO: 12 - protein, 14 aa, Artificial
  synthetic peptide
  MOD_RES (1)..(1): N′ ACETYLATED citrulline
  MOD_RES (3)..(3): 3-((2-naphthyl) alanine
  MOD_RES (6)..(6): citrulline
  MOD_RES (8)..(8): D-lysine
  MOD_RES (12)..(12): citrulline
  MOD_RES (14)..(14): AMIDATED
XRXCYXKXPYRXCR

SEQ ID NO: 13 - protein, 14 aa, Artificial
  synthetic peptide
  MOD_RES (1)..(1): N′ ACETYLATED citrulline
  MOD_RES (3)..(3): 3-((2-naphthyl) alanine
  MOD_RES (8)..(8): D-citrulline
  MOD_RES (12)..(12): citrulline
  MOD_RES (14)..(14): C′ AMIDATED
XRXCYRKXPYRXCR

SEQ ID NO: 14 - protein, 14 aa, Artificial
  synthetic peptide
  MOD_RES (1)..(1): N′ ACETYLATED
  MOD_RES (3)..(3): 3-((2-naphthyl) alanine
  MOD_RES (8)..(8): D-citrulline
  MOD_RES (11)..(12): citrulline
  MOD_RES (14)..(14): C′ AMIDATED
RRXCYRKXPYXXCR

SEQ ID NO: 15 - protein, 14 aa, Artificial
  synthetic peptide
  MOD_RES (1)..(1): N′ ACETYLATED citrulline
  MOD_RES (3)..(3): 3-((2-naphthyl) alanine
  MOD_RES (8)..(8): D-lysine
  MOD_RES (11)..(12): citrulline
  MOD_RES (14)..(14): C′ AMIDATED
XRXCYRKXPYXXCR

SEQ ID NO: 16 - protein, 14 aa, Artificial
  synthetic peptide
  MOD_RES (1)..(1): D-glutamic acid
  MOD_RES (3)..(3): 3-((2-naphthyl) alanine
  MOD_RES (8)..(8): D-lysine
  MOD_RES (12)..(12): citrulline
XRXCYRKXPYRXCR

SEQ ID NO: 17 - protein, 14 aa, Artificial
  synthetic peptide
  MOD_RES (3)..(3): 3-((2-naphthyl) alanine
  MOD_RES (8)..(8): D-lysine
  MOD_RES (12)..(12): citrulline
REXCYRKXPYRXCR

SEQ ID NO: 18 - protein, 14 aa, Artificial
  synthetic peptide
  MOD_RES (3)..(3): 3-((2-naphthyl) alanine
  MOD_RES (8)..(8): D-lysine
  MOD_RES (12)..(12): citrulline
RRXCYEKXPYRXCR

SEQ ID NO: 19 - protein, 14 aa, Artificial
  synthetic peptide
  MOD_RES (3)..(3): 3-((2-naphthyl) alanine
  MOD_RES (8)..(8): D-lysine
  MOD_RES (12)..(12): citrulline
RRXCYREXPYRXCR

SEQ ID NO: 20 - protein, 14 aa, Artificial
  synthetic peptide
  MOD_RES (3)..(3): 3-((2-naphthyl) alanine
  MOD_RES (8)..(8): D-glutamic acid
  MOD_RES (12)..(12): citrulline
RRXCYRKXPYRXCR

SEQ ID NO: 21 - protein, 14 aa, Artificial
  synthetic peptide
  MOD_RES (3)..(3): 3-((2-naphthyl) alanine
  MOD_RES (8)..(8): D-lysine
  MOD_RES (12)..(12): citrulline
RRXCYRKXPYEXCR

SEQ ID NO: 22 - protein, 14 aa, Artificial
  synthetic peptide
  MOD_RES (3)..(3): 3-((2-naphthyl) alanine
  MOD_RES (8)..(8): D-lysine
  MOD_RES (12)..(12): citrulline
RRXCYRKXPYRXCE

SEQ ID NO: 23 - protein, 14 aa, Artificial
  synthetic peptide
  MOD_RES (3)..(3): 3-((2-naphthyl) alanine
  MOD_RES (6)..(6): citrulline
  MOD_RES (8)..(8): D-glutamic acid
  MOD_RES (12)..(12): citrulline
  MOD_RES (14)..(14): C′ amidated
RRXCYXKXPYRXCR

SEQ ID NO: 24 - protein, 14 aa, Artificial
  synthetic peptide
  MOD_RES (3)..(3): 3-((2-naphthyl) alanine
  MOD_RES (6)..(6): D-glutamic acid
  MOD_RES (8)..(8): D-citrulline
  MOD_RES (12)..(12): citrulline
  MOD_RES (14)..(14): c′ amidated
RRXCYXKXPYRXCR

SEQ ID NO: 25 - protein, 14 aa, Artificial
  synthetic peptide
  MOD_RES (3)..(3): 3-((2-naphthyl) alanine
  MOD_RES (6)..(6): D-glutamic acid
  MOD_RES (8)..(8): D-glutamic acid
  MOD_RES (12)..(12): citrulline
  MOD_RES (14)..(14): c′ amidated
RRXCYXKXPYRXCR

SEQ ID NO: 26 - protein, 14 aa, Artificial
  synthetic peptide
  MOD_RES (1)..(1): D-glutamic acid
  MOD_RES (3)..(3): 3-((2-naphthyl) alanine
  MOD_RES (6)..(6): citrulline
  MOD_RES (8)..(8): D-glutamic acid
  MOD_RES (12)..(12): citrulline
  MOD_RES (14)..(14): c′ amidated
XRXCYXKXPYRXCR

SEQ ID NO: 27 - protein, 14 aa, Artificial
  synthetic peptide
  MOD_RES (3)..(3): 3-((2-naphthyl) alanine
  MOD_RES (6)..(6): citrulline
  MOD_RES (8)..(8): D-glutamic acid
  MOD_RES (10)..(10): D-glutamic acid
  MOD_RES (12)..(12): citrulline
  MOD_RES (14)..(14): c′ amidated
RRXCYXKXPXRXCR

SEQ ID NO: 28 - protein, 14 aa, Artificial
  synthetic peptide
  MOD_RES (3)..(3): 3-((2-naphthyl) alanine
  MOD_RES (6)..(6): citrulline
  MOD_RES (8)..(8): D-glutamic acid
  MOD_RES (12)..(12): D-glutamic acid
  MOD_RES (14)..(14): c′ amidated
RRXCYXKXPYRXCR

SEQ ID NO: 29 - protein, 14 aa, Artificial
  synthetic peptide
  MOD_RES (1)..(1): N′ ACETYLATED D-glutamic acid
  MOD_RES (3)..(3): 3-((2-naphthyl) alanine
  MOD_RES (6)..(6): citrulline
  MOD_RES (8)..(8): D-glutamic acid
  MOD_RES (12)..(12): citrulline
  MOD_RES (14)..(14): c′ amidated
XRXCYXKXPYRXCR

SEQ ID NO: 30 - protein, 14 aa, Artificial
  synthetic peptide
  MOD_RES (1)..(1): N′ ACETYLATED
  MOD_RES (3)..(3): 3-((2-naphthyl) alanine
  MOD_RES (6)..(6): citrulline
  MOD_RES (8)..(8): D-glutamic acid
  MOD_RES (10)..(10): D-glutamic acid
  MOD_RES (12)..(12): citrulline
  MOD_RES (14)..(14): c′ amidated
RRXCYXKXPXRXCR

SEQ ID NO: 31 - protein, 14 aa, Artificial
  synthetic peptide
  MOD_RES (1)..(1): N′ ACETYLATED
  MOD_RES (3)..(3): 3-((2-naphthyl) alanine
  MOD_RES (6)..(6): citrulline
  MOD_RES (8)..(8): D-glutamic acid
  MOD_RES (12)..(12): D-glutamic acid
  MOD_RES (14)..(14): c′ amidated
RRXCYXKXPYRXCR

SEQ ID NO: 32 - protein, 14 aa, Artificial
  synthetic peptide
  MOD_RES (1)..(1): N′ ACETYLATED
  MOD_RES (3)..(3): 3-((2-naphthyl) alanine
  MOD_RES (6)..(6): citrulline
  MOD_RES (8)..(8): D-glutamic acid
  MOD_RES (12)..(12): citrulline
  MOD_RES (14)..(14): c′ amidated
RRXCYXKXPYRXCR

SEQ ID NO: 33 - protein, 14 aa, Artificial
  synthetic peptide
  MOD_RES (1)..(1): Guanyl-arginine
  MOD_RES (3)..(3): 3-((2-naphthyl) alanine
  MOD_RES (6)..(6): citrulline
  MOD_RES (8)..(8): D-glutamic acid
  MOD_RES (12)..(12): citrulline
  MOD_RES (14)..(14): c′ amidated
XRXCYXKXPYRXCR

SEQ ID NO: 34 - protein, 14 aa, Artificial
  synthetic peptide
  MOD_RES (1)..(1): Tetramethylguanyl-arginine
  MOD_RES (3)..(3): 3-((2-naphthyl) alanine
  MOD_RES (6)..(6): citrulline
  MOD_RES (8)..(8): D-glutamic acid
  MOD_RES (12)..(12): citrulline
  MOD_RES (14)..(14): c′ amidated
XRXCYXKXPYRXCR

SEQ ID NO: 35 - protein, 13 aa, Artificial
  synthetic peptide
  MOD_RES (1)..(1): Tetramethylguanyl-arginine
  MOD_RES (2)..(2): 3-((2-naphthyl) alanine
  MOD_RES (5)..(5): citrulline
  MOD_RES (7)..(7): D-glutamic acid
  MOD_RES (11)..(11): citrulline
  MOD_RES (13)..(13): c′ amidated
XXCYXKXPYRXCR

SEQ ID NO: 36 - protein, 14 aa, Artificial
  synthetic peptide
  MOD_RES (1)..(1): 4-fluorobenzoyl-arginine
  MOD_RES (3)..(3): 3-((2-naphthyl) alanine
  MOD_RES (6)..(6): citrulline
  MOD_RES (8)..(8): D-glutamic acid
  MOD_RES (12)..(12): citrulline
  MOD_RES (14)..(14): amidated
XRXCYXKXPYRXCR

SEQ ID NO: 37 - protein, 14 aa, Artificial
  synthetic peptide
  MOD_RES (1)..(1): 2-fluorobenzoyl-arginine
  MOD_RES (3)..(3): 3-((2-naphthyl) alanine
  MOD_RES (6)..(6): citrulline
  MOD_RES (8)..(8): D-glutamic acid
  MOD_RES (12)..(12): citrulline
  MOD_RES (14)..(14): c′ amidated
XRXCYXKXPYRXCR

SEQ ID NO: 38 - protein, 13 aa, Artificial
  synthetic peptide
  MOD_RES (1)..(1): 5-aminopentanoyl-arginine
  MOD_RES (2)..(2): 3-((2-naphthyl) alanine
  MOD_RES (5)..(5): citrulline
  MOD_RES (7)..(7): D-glutamic acid
  MOD_RES (11)..(11): citrulline
  MOD_RES (13)..(13): c′ amidated
XXCYXKXPYRXCR

SEQ ID NO: 39 - protein, 14 aa, Artificial
  synthetic peptide
  MOD_RES (1)..(1): 2-desamino-arginyl
  MOD_RES (3)..(3): 3-((2-naphthyl) alanine
  MOD_RES (6)..(6): citrulline
  MOD_RES (8)..(8): D-glutamic acid
  MOD_RES (12)..(12): citrulline
  MOD_RES (14)..(14): c′ amidated
XRXCYXKXPYRXCR

SEQ ID NO: 40 - protein, 13 aa, Artificial
  synthetic peptide
  MOD_RES (1)..(1): Guanyl-arginine
  MOD_RES (2)..(2): 3-((2-naphthyl) alanine
  MOD_RES (5)..(5): citrulline
  MOD_RES (7)..(7): D-glutamic acid
  MOD_RES (11)..(11): citrulline
  MOD_RES (13)..(13): c′ amidated
XXCYXKXPYRXCR

SEQ ID NO: 41 - protein, 13 aa, Artificial
  synthetic peptide
  MOD_RES (1)..(1): Succinyl-arginine
  MOD_RES (2)..(2): 3-((2-naphthyl) alanine
  MOD_RES (5)..(5): citrulline
  MOD_RES (7)..(7): D-glutamic acid
  MOD_RES (11)..(11): citrulline
  MOD_RES (13)..(13): c′ amidated
XXCYXKXPYRXCR

SEQ ID NO: 42 - protein, 13 aa, Artificial
  synthetic peptide
  MOD_RES (1)..(1): Glutaryl-arginine
  MOD_RES (2)..(2): 3-((2-naphthyl) alanine
  MOD_RES (5)..(5): citrulline
  MOD_RES (7)..(7): D-glutamic acid
  MOD_RES (11)..(11): citrulline
  MOD_RES (13)..(13): c′ amidated
XXCYXKXPYRXCR

SEQ ID NO: 43 - protein, 14 aa, Artificial
  synthetic peptide
  MISC_FEATURE (1)..(1): desaminoTMG-APA (formula IV in the specification)
  MOD_RES (3)..(3): 3-((2-naphthyl) alanine
  MOD_RES (6)..(6): citrulline
  MOD_RES (8)..(8): D-glutamic acid
  MOD_RES (12)..(12): citrulline
  MOD_RES (14)..(14): c′ amidated
XRXCYXKXPYRXCR

SEQ ID NO: 44 - protein, 14 aa, Artificial
  synthetic peptide
  MISC_FEATURE (1)..(1): R-CH2 - formula (V) in the specification
  MOD_RES (3)..(3): 3-((2-naphthyl) alanine
  MOD_RES (6)..(6): citrulline
  MOD_RES (8)..(8): D-glutamic acid
  MOD_RES (12)..(12): citrulline
  MOD_RES (14)..(14): c′ amidated
XRXCYXKXPYRXCR

SEQ ID NO: 45 - protein, 13 aa, Artificial
  synthetic peptide
  MOD_RES (2)..(2): 3-((2-naphthyl) alanine
  MOD_RES (5)..(5): citrulline
  MOD_RES (7)..(7): D-glutamic acid
  MOD_RES (11)..(11): citrulline
  MOD_RES (13)..(13): c′ amidated
RXCYXKXPYRXCR

SEQ ID NO: 46 - protein, 14 aa, Artificial
  synthetic peptide
  MOD_RES (1)..(1): tetramethylguanyl-arginine
  MOD_RES (3)..(3): 3-((2-naphthyl) alanine
  MOD_RES (6)..(6): citrulline
  MOD_RES (8)..(8): D-citrulline
  MOD_RES (12)..(12): citrulline
  MOD_RES (14)..(14): C′ AMIDATED
XRXCYXKXPYRXCR

SEQ ID NO: 47 - protein, 14 aa, Artificial
  synthetic peptide
  MOD_RES (1)..(1): 6-aminohexanoyl-arginine
  MOD_RES (3)..(3): 3-((2-naphthyl) alanine
  MOD_RES (6)..(6): citrulline
  MOD_RES (8)..(8): D-citrulline
  MOD_RES (12)..(12): citrulline
  MOD_RES (14)..(14): C′ AMIDATED
XRXCYXKXPYRXCR

SEQ ID NO: 48 - protein, 14 aa, Artificial
  synthetic peptide
  MOD_RES (1)..(1): 6-aminohexanoyl-arginine
  MOD_RES (3)..(3): 3-((2-naphthyl) alanine
  MOD_RES (8)..(8): D-lysine
  MOD_RES (12)..(12): citrulline
XRXCYRKXPYRXCR

SEQ ID NO: 49 - protein, 14 aa, Artificial
  synthetic peptide
  MOD_RES (3)..(3): 3-((2-naphthyl) alanine
  MOD_RES (6)..(6): citrulline
  MOD_RES (8)..(8): D-lysine
  MOD_RES (12)..(12): citrulline
  MOD_RES (14)..(14): C′ amidated
RRXCYXRXPYRXCR

SEQ ID NO: 50 - protein, 14 aa, Artificial
  synthetic peptide
  MOD_RES (1)..(1): N′ ACETYLATED
  MOD_RES (3)..(3): 3-((2-naphthyl) alanine
  MOD_RES (6)..(6): citrulline
  MOD_RES (8)..(8): D-lysine
  MOD_RES (12)..(12): citrulline
  MOD_RES (14)..(14): C′ amidated
RRXCYXRXPYRXCR

SEQ ID NO: 51 - protein, 14 aa, Artificial
  SYNTHETIC PEPTIDE
  MOD_RES (1)..(1): N′ ACETYLATED
  MOD_RES (3)..(3): 3-((2-naphthyl) alanine
  MOD_RES (6)..(6): citrulline
  MOD_RES (8)..(8): D-lysine
  MOD_RES (12)..(12): citrulline
  MOD_RES (14)..(14): C′ AMIDATED
RRXCYXKXPYRXCR

SEQ ID NO: 52 - protein, 14 aa, Artificial
  synthetic peptide
  MOD_RES (1)..(1): N′ ACETYLATED
  MOD_RES (3)..(3): 3-((2-naphthyl) alanine
  MOD_RES (8)..(8): D-citrulline
  MOD_RES (12)..(12): citrulline
  MOD_RES (14)..(14): C′ AMIDATED
RRXCYRKXPYRXCR

SEQ ID NO: 53 - protein, 14 aa, Artificial
  synthetic peptide
  MOD_RES (1)..(1): 4-fluorobenzoyl-arginine
  MOD_RES (3)..(3): 3-((2-naphthyl) alanine
  MOD_RES (6)..(6): citrulline
  MOD_RES (8)..(8): D-glutamic acid
  MOD_RES (12)..(12): citrulline
  MISC_FEATURE (14)..(14): derivatization by a NH-methyl group
XRXCYXKXPYRXCR

SEQ ID NO: 54 - protein, 14 aa, Artificial
  synthetic peptide
  MOD_RES (1)..(1): 4-fluorobenzoyl-arginine
  MOD_RES (3)..(3): 3-((2-naphthyl) alanine
  MOD_RES (6)..(6): citrulline
  MOD_RES (8)..(8): D-glutamic acid
  MOD_RES (12)..(12): citrulline
  MISC_FEATURE (14)..(14): derivatization by a NH-ethyl group
XRXCYXKXPYRXCR

SEQ ID NO: 55 - protein, 14 aa, Artificial
  synthetic peptide
  MOD_RES (1)..(1): 4-fluorobenzoyl-arginine
  MOD_RES (3)..(3): 3-((2-naphthyl) alanine
  MOD_RES (6)..(6): citrulline
  MOD_RES (8)..(8): D-glutamic acid
  MOD_RES (12)..(12): citrulline
  MISC_FEATURE (14)..(14): derivatization by NH-isopropyl
XRXCYXKXPYRXCR

SEQ ID NO: 56 - protein, 14 aa, Artificial
  synthetic peptide
  MOD_RES (1)..(1): 4-fluorobenzoyl-arginine
  MOD_RES (3)..(3): 3-((2-naphthyl) alanine
  MOD_RES (6)..(6): citrulline
  MOD_RES (8)..(8): D-glutamic acid
  MOD_RES (12)..(12): citrulline
  MISC_FEATURE (14)..(14): derivatization with a tyramine residue
XRXCYXKXPYRXCR

SEQ ID NO: 57 - protein, 14 aa, Artificial
  synthetic peptide
  MOD_RES (3)..(3): 3-((2-naphthyl) alanine
  MOD_RES (8)..(8): D-lysine
  MOD_RES (12)..(12): citrulline
ARXCYRKXPYRXCR

SEQ ID NO: 58 - protein, 14 aa, Artificial
  synthetic peptide
  MOD_RES (3)..(3): 3-((2-naphthyl) alanine
  MOD_RES (8)..(8): D-lysine
  MOD_RES (12)..(12): citrulline
RRXCYAKXPYRXCR

SEQ ID NO: 59 - protein, 14 aa, Artificial
  synthetic peptide
  MOD_RES (3)..(3): 3-((2-naphthyl) alanine
  MOD_RES (8)..(8): D-lysine
  MOD_RES (12)..(12): citrulline
RRXCYRAXPYRXCR

SEQ ID NO: 60 - protein, 14 aa, Artificial
  synthetic peptide
  MOD_RES (3)..(3): 3-((2-naphthyl) alanine
  MOD_RES (8)..(8): D-alanine
  MOD_RES (12)..(12): citrulline
RRXCYRKXPYRXCR

SEQ ID NO: 61 - protein, 14 aa, Artificial
  synthetic peptide
  MOD_RES (3)..(3): 3-((2-naphthyl) alanine
  MOD_RES (8)..(8): D-lysine
  MOD_RES (12)..(12): citrulline
RRXCYRKXAYRXCR

SEQ ID NO: 62 - protein, 14 aa, Artificial
  synthetic peptide
  MOD_RES (3)..(3): 3-((2-naphthyl) alanine
  MOD_RES (8)..(8): D-lysine
  MOD_RES (12)..(12): citrulline
RRXCYRKXPARXCR

SEQ ID NO: 63 - protein, 14 aa, Artificial
  synthetic peptide
  MOD_RES (3)..(3): 3-((2-naphthyl) alanine
  MOD_RES (8)..(8): D-lysine
  MOD_RES (12)..(12): citrulline
RRXCYRKXPYAXCR

SEQ ID NO: 64 - protein, 14 aa, Artificial
  synthetic peptide
  MOD_RES (1)..(1): citrulline
  MOD_RES (3)..(3): 3-((2-naphthyl) alanine
  MOD_RES (8)..(8): D-lysine
  MOD_RES (12)..(12): citrulline
XRXCYRKXPYRXCR

SEQ ID NO: 65 - protein, 14 aa, Artificial
  synthetic peptide
  MOD_RES (3)..(3): 3-((2-naphthyl) alanine
  MOD_RES (6)..(6): citrulline
  MOD_RES (8)..(8): D-lysine
  MOD_RES (12)..(12): citrulline
RRXCYXKXPYRXCR

SEQ ID NO: 66 - protein, 14 aa, Artificial
  synthetic peptide
  MOD_RES (3)..(3): 3-((2-naphthyl) alanine
  MOD_RES (6)..(6): citrulline
  MOD_RES (8)..(8): D-lysine
  MOD_RES (12)..(12): citrulline
  MOD_RES (14)..(14): C′ AMIDATED
RRXCYXKXPYRXCR

SEQ ID NO: 67 - protein, 14 aa, Artificial
  synthetic peptide
  MOD_RES (3)..(3): 3-((2-naphthyl) alanine
  MOD_RES (7)..(7): citrulline
  MOD_RES (8)..(8): D-lysine
  MOD_RES (12)..(12): citrulline
RRXCYRXXPYRXCR

SEQ ID NO: 68 - protein, 14 aa, Artificial
  synthetic peptide
  MOD_RES (3)..(3): 3-((2-naphthyl) alanine
  MOD_RES (6)..(6): citrulline
  MOD_RES (8)..(8): D-citrulline
  MOD_RES (12)..(12): citrulline
  MOD_RES (14)..(14): C′ AMIDATED
RRXCYXKXPYRXCR

SEQ ID NO: 69 - protein, 14 aa, Artificial
  synthetic peptide
  MOD_RES (3)..(3): 3-((2-naphthyl) alanine
  MOD_RES (8)..(8): D-lysine
  MOD_RES (12)..(12): citrulline
RRXCYRKXPYRXCR

SEQ ID NO: 70 - protein, 14 aa, Artificial
  synthetic peptide
  MOD_RES (3)..(3): 3-((2-naphthyl) alanine
  MOD_RES (6)..(6): citrulline
  MOD_RES (8)..(8): D-citrulline
  MOD_RES (12)..(12): citrulline
RRXCYXKXPYRXCR

SEQ ID NO: 71 - protein, 14 aa, Artificial
  synthetic peptide
  MOD_RES (3)..(3): 3-((2-naphthyl) alanine
  MOD_RES (8)..(8): D-citrulline
  MOD_RES (12)..(12): citrulline
RRXCYRKXPYRXCR

SEQ ID NO: 72 - protein, 14 aa, Artificial
  synthetic peptide
  MOD_RES (1)..(1): citrulline
  MOD_RES (3)..(3): 3-((2-naphthyl) alanine
  MOD_RES (8)..(8): D-citrulline
  MOD_RES (12)..(12): citrulline
  MOD_RES (14)..(14): C′ Amidated
XRXCYRKXPYRXCR

SEQ ID NO: 73 - DNA, 20 bp, Artificial sequence
  Single strand DNA oligonucleotide
gaaccctgtttccgtgaaga

SEQ ID NO: 74 - DNA, 20 bp, Artificial sequence
  Single strand DNA oligonucleotide
cttgtccgtcatgcttctca

SEQ ID NO: 75 - DNA, 21 bp, Artificial sequence
  Single strand DNA oligonucleotide
gtctgttgttgttcttcagcc

SEQ ID NO: 76 - DNA, 20 bp, Artificial sequence
  Single strand DNA oligonucleotide
atgcccatgccgattcttcg

SEQ ID NO: 77 - DNA, 20 bp, Artificial sequence
  Single strand DNA oligonucleotide
gataacggaggctgggatgc

SEQ ID NO: 78 - DNA, 20 bp, Artificial sequence
  Single strand DNA oligonucleotide
tcacttgtggcccagatagg

SEQ ID NO: 79 - DNA, 20 bp, Artificial sequence
  Single strand DNA oligonucleotide
ttgccctctgtgccacagat

SEQ ID NO: 80 - DNA, 20 bp, Artificial sequence
  Single strand DNA oligonucleotide
tcaggttcaggccttgcact

SEQ ID NO: 81 - DNA, 22 bp, Artificial sequence
  Single strand DNA oligonucleotide
tagcagcacataatggtttgtg

SEQ ID NO: 82 - DNA, 22 bp, Artificial sequence
  Single strand DNA oligonucleotide
tagcagcacgtaaatattggcg

SEQ ID NO: 83 - DNA, 21 bp, Artificial sequence
  Single strand DNA oligonucleotide
ggacaggactgaacgtcttgc

SEQ ID NO: 84 - DNA, 20 bp, Artificial sequence
  Single strand DNA oligonucleotide
caacacttcgtggggtcctt

SEQ ID NO: 85 - DNA, 23 bp, Artificial sequence
  Single strand DNA oligonucleotide
ggggctttatctaactcgctgta

SEQ ID NO: 86 - DNA, 21 bp, Artificial sequence
  Single strand DNA oligonucleotide
tatgggcaaagtttcgtggat

(19) United States
(12) Patent Application Publication
(10) Pub. No.: US 2020/0282009 A1
(43) Pub. Date: Sep. 10, 2020

(54) METHODS OF TREATING DISEASES ASSOCIATED WITH VASCULAR SMOOTH MUSCLE CELL PROLIFERATION

(71) Applicants: BioLineRx Ltd., ModiIn (IL); Biokine Therapeutics Ltd., Nes Ziona (IL)

(72) Inventor: Amnon PELED, Tel-Aviv (IL)

(73) Assignees: BioLineRx Ltd., ModiIn (IL); Biokine Therapeutics Ltd., Nes Ziona (IL)

(21) Appl. No.: 16/646,594

(22) PCT Filed: Sep. 12, 2018
(86) PCT No.: PCT/IL2018/051023

§ 371 Date: Mar. 12, 2020

Related U.S. Application Data
(60) Provisional application No. 62/557,197, filed on Sep. 12, 2017.

(51) Int. Cl. A61K 38/10

(52) U.S. Cl. CPC A61K 38/10; A61K 9/0024

(57) ABSTRACT

A method of treating a disease associated with vascular smooth muscle cell proliferation in a subject in need thereof is provided with the proviso that the disease is not cancer. The method comprises administering to the subject a therapeutically effective amount of a CXCR4-antagonistic peptide comprising SEQ ID NO: 1, thereby treating the disease.

### FIELD AND BACKGROUND OF THE INVENTION

The present invention, in some embodiments thereof, relates methods of treating diseases associated with vascular smooth muscle cell proliferation.

c-myc is a master regulatory factor of cell proliferation. Pathologic activation of c-myc plays a central role in neoplastic disease pathogenesis by the coordinated upregulation of a transcriptional program influencing cell division, metabolic adaptation and survival. Amplification of c-myc is among the most common genetic alterations observed in cancer genomes. Validation of c-myc as a therapeutic target in cancer is supported by numerous lines of experimental evidence.

A growing body of evidence indicates that c-Myc plays a pivotal role also in cardiovascular diseases. Indeed, alterations of the basal machinery of the cell and perturbations of c-Myc-dependent signaling network are involved in the pathogenesis of certain cardiovascular disorders. Down-regulation of c-Myc induced by intervention with antioxidants or by antisense technology may protect the integrity of the arterial wall as well as neoplastic tissues (reviewed by Napoli et al. 2002 Neoplasia 4(3):185-190).

Nevertheless, a therapeutic approach to target c-Myc has remained elusive. The absence of a clear ligand-binding domain establishes a formidable obstacle toward direct inhibition, which is a challenging feature shared among many compelling transcriptional targets. C-Myc functions as a DNA-binding transcriptional activator upon heterodimerization with another basic-helic-turn-helix leucine zipper, transcription factor, Max. High resolution structures of the complex fail to identify a hydrophobic involution compatible with the positioning of an organic small molecule Reviewed in Delmore et al. Cell 146 904-917, 2011).

4F-benzoyl-TN14003 (also known as BKT140, hereinafter BL-8040), is a 14-residue bio stable synthetic peptide developed as a specific CXCR4 antagonist. It has been shown that BL-8040 binds the CXCR4 receptor with high affinity and long receptor occupancy (Peled A, Wald O, Burger J. Development of novel CXCR4-based therapeutics. Expert Opin Investig Drugs. 2012 March; 21(3):341-53). The peptide exhibits toxic effects on CXCR4-expressing malignancies including: multiple myeloma, acute myeloid leukemia (AML), chronic myeloid leukemia, non-small cell lung cancer and metastatic breast cancer (8,16-18). Currently, BL-8040 is in phase 2 clinical trials for AML and pancreatic cancer. The present inventor has previously shown that over-expression of CXCR4 on tumor cells and simulation of cells with CXCL12 leads to up-regulation of miR-15a/16-1 and consequently down regulation of their target genes BCL-2, MCL-1, and cyclin D1. Furthermore, overexpression of CXCR4 in these cells increases tumorogenesis and shift their oncogenic dependency from BCL2 to CXCR4. The present inventor further showed that that CXCR4 binding agent BL8040 has a similar effect on miR-15a/16-1. Thus, BL8040 induced apoptosis of tumor cell by inhibiting survival signals while keeping apoptotic gene expression low both in vitro and in vivo.

### Additional Related Art

Sheng et al. Cardiovasc J Afr. 2011 November-December; 22(6):313-8.
Zernecke et al. Circ Res. 2005; 96:784-91.
Bot et al. J Mol Cell Cardiol. 2014 September; 74: 44-52.
WO2017/021963

### SUMMARY OF THE INVENTION

According to an aspect of some embodiments of the present invention there is provided a method of treating a disease associated with vascular smooth muscle cell proliferation in a subject in need thereof, with the proviso that the disease is not cancer, the method comprising administering to the subject a therapeutically effective amount of a CXCR4-antagonistic peptide comprising SEQ ID NO: 1, thereby treating the disease.

According to an aspect of some embodiments of the present invention there is provided a composition comprising a CXCR4-antagonistic peptide comprising SEQ ID NO: 1 for the treatment of a disease associated with vascular smooth muscle cell proliferation in a subject in need thereof with the proviso that the disease is not cancer.

According to an aspect of some embodiments of the present invention there is provided a device for cardiac delivery comprising a CXCR4-antagonistic peptide comprising SEQ ID NO. 1.

According to some embodiments of the invention, the disease is a cardiac disease.

According to some embodiments of the invention, the cardiac disease is selected from the group consisting of cardiac hypertrophy, transplant arteriosclerosis, atherosclerosis, angioplasty restenosis, ventricular stenosis or cardiac vein bypass stenosis.

According to some embodiments of the invention, the disease is selected from the group consisting of essential hypertension, thrombosis, stenosis, ventricular stenosis, diabetic macroangiopathy, myocardial infarction, stroke, vascular dementia, intimal hyperplasia, restenosis, angioplasty restenosis, cardiac vein bypass stenosis, transplant arteriosclerosis and atherosclerosis.

According to some embodiments of the invention, the administering comprises local administering.

According to some embodiments of the invention, the composition is formulated for local administration.

According to some embodiments of the invention, the device is a stent or a catheter.

Unless otherwise defined, all technical and/or scientific terms used herein have the same meaning as commonly understood by one of ordinary skill in the art to which the invention pertains. Although methods and materials similar or equivalent to those described herein can be used in the practice or testing of embodiments of the invention, exemplary methods and/or materials are described below. In case of conflict, the patent specification, including definitions, will control. In addition, the materials, methods, and examples are illustrative only and are not intended to be necessarily limiting.

### BRIEF DESCRIPTION OF THE SEVERAL VIEWS OF THE DRAWING(S)

Some embodiments of the invention are herein described, by way of example only, with reference to the accompanying drawings. With specific reference now to the drawings in detail, it is stressed that the particulars shown are by way of example and for purposes of illustrative discussion of embodiments of the invention. In this regard, the description taken with the drawings makes apparent to those skilled in the art how embodiments of the invention may be practiced.

In the drawings:

FIGS. 1A-E show CXCR4 expression in NB tumors and tumor derived cell lines. (FIG. 1A) Tissue array including 13 samples from patients with NB stained for CXCR4. Scale bar 100 μm. (FIG. 1B) Table of tissue array tumors and scoring intensities. (FIG. 1C) Relative mRNA levels of CXCR4 and CXCL12 in human NB cell lines SK-N-BE(2), ShyY-SY5Y and MHH-NB-11 assessed by qRT-PCR. MRNA levels were normalized to HPRT. Data is presented as mean±SD from triplicates (* p<0.05). (FIG. 1D) CXCR4 expression in SK-N-BE(2), Shy-SY-5Y and MHH-NB-11 cells evaluated by FACS. Green line represents IgG control antibody, purple line represents 12G5 monoclonal antibody external staining (left panel), internal staining (right panel). (FIG. 1E) CXCL12 levels in SK-N-BE(2) and Shy-SY5Y cells in-vitro was measured by ELISA.

FIGS. 2A-I show that proliferation and viability of NB cells is CXCR4 dependent. (FIG. 2A) SK-N-BE(2) and Shy-SY5Y cells seeded at 10∧5 cells per well into a 24 well plate, incubated with increasing concentrations of CXCL12 (0, 50, 250 and 500 ng/ml). On days 2 and 4 cells were harvested, viable cells counted using PI staining and FACS analysis. (FIG. 2B) CXCL12 enhances colony formation of SK-N-BE(2) cells in-vitro. Cells plated in triplicates in soft agar with CXCL12 (0 and 100 ng/ml) for 14 days. Following 14 days, colony size and number measured by image J software. (FIG. 2C) The NB cell lines SK-N-BE(2) and ShY-SY5Y treated with increased concentrations of BL-8040 (0, 2.5, 5, 10 and 20 μM) or AMD3100 (10 and 20 μM) for 24 hours. Viable cells counted using PI staining and FACS analysis. (FIG. 2D) CXCR4 antibodies or BL-8040 reduced colony formation of SK-N-BE(2) and Shy-SY5Y cells. Cells plated in triplicates in soft agar with anti-CXCR4(1 ug/ml) or BL-8040 (20 μM) for 14 days. Following 14 days, colony number was measured by image J software. (FIG. 2E) Relative mRNA levels of CXCR4 following lentivirus transduction with specific CXCR4 expressing plasmid and control cells assessed by qPCR. MRNA levels were normalized to HPRT. Data represent mean±SD (* p<0.05). (FIG. 2F) FACS analysis of CXCR4 surface expression on native SK-N-BE(2) cell line (green) and on SK-N-BE(2) cells transduced to overexpress CXCR4 (purple). (FIG. 2G) SK-N-BE(2) and SK-N-BE(2)-CXCR4 cells seeded at 10∧4 cells per well into a chamber and cell number determined using the xCELLignece system. (FIG. 2H) SK-N-BE(2) and SK-N-BE(2)-CXCR4 cells seeded in soft agar in triplicates. Following 14 days, colonies photographed and counted. (FIG. 2I) SK-N-BE(2) and SK-N-BE(2)-CXCR4 cells treated with increased concentrations of BL-8040 (0, 10 and 20 μM) for 24 hours. Viable cells counted using PI staining and FACS analysis. Data is presented as mean±SD from triplicates (* p<0.05).

FIGS. 3A-J show that BL-8040 inhibits NB tumor growth in vivo. SK-N-BE(2) cells were injected into the left adrenal gland of mice. (FIG. 3A) Representative T2W tumor anatomical axial images acquired on days 19, 26 and 29. (FIG. 3B) Mice scanned bi-weekly and tumor volumes (mm³) measured from T2W MRI images as a function of days post tumor detection (n=19). (FIG. 3C) CXCR4 and CXCL12 immunostaining of paraffin-embedded tissue sections derived from SK-N-BE(2) tumors. Scale bar 100 μm. (FIG. 3D) Relative CXCR4 mRNA levels in the SK-N-BE(2) cell line in-vitro and following tumor formation (n=5) assessed by RT-PCR. Data represent mean±SD, all normalized against HPRT expression (* p<0.05). (FIG. 3E) CXCL12 levels in SK-N-BE(2) cells in-vitro and following tumor formation was measured by ELISA. The results represent the average of triplicates±SD (*p<0.05). (FIG. 3F) SK-N-BE(2) and SK-N-BE(2)-CXCR4 cells were injected, 21 days following injection tumors were harvested. Shown as tumor weight±SE (*P<0.05) in mg. (FIGS. 3G-H) Three days after SK-N-BE(2) cell injection, BL-8040 was administered subcutaneously at a dose of 400 μg per injection daily for 35 days. (FIG. 3G) Tumor volume (mm³) for each individual mouse, as measured from T2W MRI images as a function of days post cell inoculation in control (n=6) and BL-8040 (n=7) treated mice. The gray area indicates BL-8040 treatment period. (FIG. 3H) Kaplan-Meier survival analysis for control versus treated groups, P<0.001 by mantel-cox test. (FIGS. 3I-J) Seven days after cell injection, BL-8040 was administered subcutaneously 400 μg per injection daily for 14 days. (FIG. 3I) Average tumor weight±SE (*P<0.05) shown in mg of tumors harvested from control (n=7) and treated (n=8) mice on day 21 from two experiments. (FIG. 3J) Quantification of Ki67 positive cells in sections of control and BL-8040 treated SK-N-BE(2) xenografts. Data is presented as mean±SD (* P<0.05) control (n=5), BL-8040 (n=5);

FIGS. 4A-D show the identification of miR-15a and miR-16-1 as target microRNAs regulated by the CXCR4 inhibitor BL-8040. (FIG. 4A) Heatmap of genes differentially expressed by xenograft SK-N-BE(2) tumors treated with BL-8040 (n=3) versus control (n=3). (FIG. 4B) Validation of miR-15a and miR-16-1 expression in extracted RNA from control (n=9) versus BL-8040 treated (n=9) tumors by qPCR. Data represent the mean from two different experiments±SD, all normalized against RNU44 expression (* p<0.05). (FIG. 4C) Relative mRNA levels of BCL-2 and CCND1 from SK-N-BE(2) xenograft tumors from control (n=9) versus BL-8040 treated (n=9) assessed by qPCR. Data represent the mean from two different experiments±SD, all normalized against HPRT expression (* p<0.05). (FIG. 4D) Cyclin D1 and BCL-2 immunostaining of paraffin-embedded tissue sections derived from SK-N-BE(2) tumors treated with BL-8040 compared to controls. Scale bar 20 μm.

FIGS. 5A-G show that miR-15a and miR-16-1 are regulated by the CXCR4 inhibitor BL-8040 in NB cells in-vitro. (FIG. 5A) Relative levels of miR-15a and miR-16-1 in SK-N-BE(2) and Shy-SY5Y cells treated with BL-8040 (20 μM) for 24 hours assessed by qPCR. MiR levels were normalized against RNU44 expression, data represent mean±SD (* p<0.05). (FIG. 5B) Relative levels of BCL-2 and CCND1 in SK-N-BE(2) and Shy-SY5Y cells treated with BL-8040 (20 μM) for 24 hours assessed by qPCR. MRNA levels were normalized against HPRT expression, data represent mean±SD (* p<0.05). (FIGS. 5C-D) Western blot analysis and quantification of BCL-2, cycD1 and β-actin in SK-N-BE(2) and Shy-Sy5Y cells treated with BL-8040 (20 μM) for 24 hours. (FIG. 5G) Western blot analysis of pERK and β-actin in SK-N-BE(2) and Shy-SY5Y cells treated with BL-8040 (20 μM) for 24 hours cropped from original gel. (FIG. 5E) Viability of SK-N-BE(2) cells and Shy-SY5Y 72 hours following transfection with miR-NC, miR-15a, miR-16-1 and both miR-15a and miR-16-1. Cells were counted using PI staining and FACS analysis. Data is presented as mean±SD from triplicates (* p<0.05). (FIG. 5F) 48 hours following transfection with antagomiR-NC or both antago miR-15a and antago miR-16-1, SK-N-BE(2) and Shy-SY5Y cells were treated with BL-8040 (20 μM) for 48 hours. Viability of cells was determined using PI staining and FACS analysis. Data is presented as mean±SD from triplicates (* p<0.05).

FIGS. 6A-I show that the CXCR4 receptor upregulates miR-15a and miR-16-1 expression. (FIG. 6A) Relative levels of miR-15a and miR-16-1 in SK-N-BE(2) and SK-N-BE(2)-CXCR4 assessed by qPCR. MiR levels were normalized against RNU44 expression. Data represent mean±SD (* p<0.05). (FIG. 6B) Relative mRNA levels of BCL-2 and CCND1 in SK-N-BE(2) and SK-N-BE(2)-CXCR4 assessed by qPCR. Data represent the mean±SD, all normalized against HPRT expression (* p<0.05). (FIG. 6C) Western blot analysis of BCL-2 and Cyclin D1 in SK-N-BE(2) and SK-N-BE(2)-CXCR4 cells. (FIGS. 6D-EC-D) Relative levels of miR-15a and miR-16-1 in RPMI and PC3 versus RPMI-CXCR4 and PC3-CXCR4 cells assessed by qPCR. MiR levels were normalized against RNU44 expression. Data represent mean±SD (* p<0.05). (FIG. 6F) Western blot analysis of pERK in SK-N-BE(2) and SK-N-BE(2)-CXCR4 cells. (FIG. 6G) SK-N-BE (2) and SK-N-BE (2)-CXCR4 cells were treated with increased concentrations of ABT-199 (0, 0.01, 0.1, 1 and 10 μM) for 24 hours. Viable cells were counted using PI staining and FACS analysis. Data is presented as mean±SD from triplicates (* p<0.05). (FIG. 6H) Relative mRNA levels of c-myc in SK-N-BE(2) and SK-N-BE(2)-CXCR4 assessed by qPCR. Data represent the mean±SD, all normalized against HPRT expression (* p<0.05). (FIG. 6I) Relative levels of c-myc in SK-N-BE(2) and Shy-SY5Y cells treated with BL-8040 (20 μM) for 24 hours assessed by qPCR. MRNA levels were normalized against HPRT expression, data represent mean±SD (* p<0.05).

FIG. 6J is a graph showing FACS analysis of BCL-2 expression on the native SK-N-BE(2) cell line (purple) and on SK-N-BE(2) cells transduced to overexpress CXCR4 (green).

FIGS. 7A-B is a schematic illustration of a proposed model. (FIG. 7A) Activation of CXCR4 by overexpression of the receptor induces two important separate signaling pathways. On the one hand, there is an upregulation of miR-15a/16-1 leading to the downregulation of target genes such as BCL-2 and CCND1. On the other hand, the MAPK signaling cascade is activated. This process shifts the cells dependency leading to the oncogenic addiction on CXCR4 resulting in enhanced cell survival. (FIG. 7B) In the case of inhibition of CXCR4 by inhibitors such as BL-8040, miR-15a/16-1 are upregulated leading to reduction in BCL-2 and CCND1 survival signals. Nonetheless, MAPK signaling pathway is repressed leading to cell death.

FIG. 8A is a graph showing the relative levels of miR-15a and miR-16-1 in SK-N-BE(2) cells transfected with miR-NC, miR-15a, miR-16-1 and both miR-15a and miR-16-1 assessed by qPCR. MiR levels were normalized against RNU44 expression.

FIG. 8B is a graph showing the relative levels of BCL-2 and CCND1 in SK-N-BE(2) cells transfected with miR-NC, miR-15a, miR-16-1 and both miR-15a and miR-16-1 assessed by qPCR. MRNA levels were normalized against HPRT expression. Data represent mean±SD (* p<0.05). Data is presented as mean±SD from triplicates (* p<0.05).

FIG. 8C is a graph showing the relative levels of miR-15a and miR-16-1 in Shy-SY5Y cells transfected with miR-NC, miR-15a, miR-16-1 and both miR-15a and miR-16-1 assessed by qPCR. MiR levels were normalized against RNU44 expression.

FIGS. 9A-B are graphs showing the relative levels of miR-15a and miR-16-1 in SK-N-BE(2) cells transfected with antago miR-NC or antago miR-15a and antago miR-16-1 assessed by RT-PCR. MicroRNA levels were normalized against RNU44 expression.

### DESCRIPTION OF SPECIFIC EMBODIMENTS OF THE INVENTION

The present invention, in some embodiments thereof, relates to methods of treating diseases associated with vascular smooth muscle cell proliferation.

Before explaining at least one embodiment of the invention in detail, it is to be understood that the invention is not necessarily limited in its application to the details set forth in the following description or exemplified by the Examples. The invention is capable of other embodiments or of being practiced or carried out in various ways.

A growing body of evidence indicates that c-Myc can play a pivotal role both in neoplasia and cardiovascular diseases. Indeed, alterations of the basal machinery of the cell and perturbations of c-Myc-dependent signaling network are involved in the pathogenesis of certain cardiovascular disorders. However, c-Myc inhibition remains elusive and to-date there is no clinically advanced therapy that targets c-Myc.

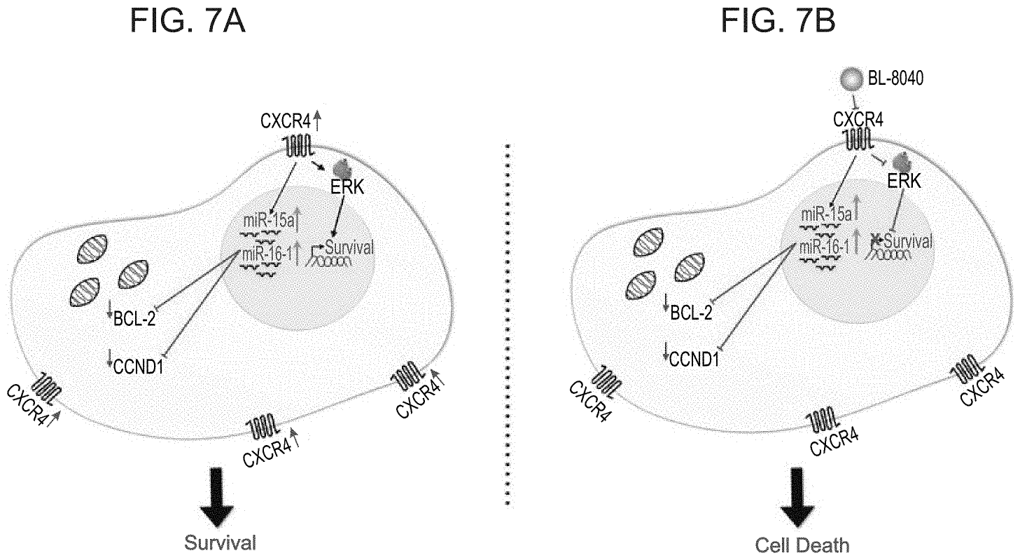

Whist reducing embodiments of the invention to practice, the present inventor has uncovered that inhibition of CXCR4 using the peptide inhibitor BL8040 can block survival signals (by upregulating miR-15a/16-1 and consequently down regulation of their target genes BCL-2, MCL-1, and cyclin D1) and in parallel also represses c-Myc signaling pathway leading to cell death (as schematically illustrated in FIGS. 7A-B).

Hence, it is now contemplated that BL-8040 can be effectively used in the treatment of c-Myc-related diseases because of its dual effect on cell survival and cell death.

Thus, according to an aspect of the invention there is provided a method of treating a disease associated with vascular smooth muscle cell proliferation in a subject in need thereof, with the proviso that the disease is not cancer, the method comprising administering to the subject a therapeutically effective amount of a CXCR4-antagonistic peptide comprising SEQ ID NO: 1, thereby treating the disease.

Alternatively, there is provided a composition comprising a CXCR4-antagonistic peptide comprising SEQ ID NO: 1 for the treatment of a disease associated with vascular smooth muscle cell proliferation in a subject in need thereof with the proviso that the disease is not cancer.

As used, herein “a disease associated with vascular smooth muscle cell proliferation” refers to a disease which onset or progression depends on vascular smooth muscle cell proliferation, which is governed by c-Myc signaling.

Methods of analyzing c-Myc activity are well known in the art (e.g., using c-Myc reporter kit available from Qiagen).

According to a specific embodiment, the disease is a cardiovascular or coronary heart disease such as cardiac hypertrophy, transplant arteriosclerosis, atherosclerosis, angioplasty restenosis, ventricular stenosis or cardiac vein bypass stenosis.

According to a specific embodiment, the disease is selected from the group consisting of essential hypertension, thrombosis, stenosis, ventricular stenosis, diabetic macroangiopathy, myocardial infarction, stroke, vascular dementia, intimal hyperplasia, restenosis, angioplasty restenosis, cardiac vein bypass stenosis, transplant arteriosclerosis and/or atherosclerosis.

According to a specific embodiment, the disease is a cardiovascular disease selected from the group consisting of atherosclerosis, thrombosis, myocardial infarction, stroke, congestive heart failure, dilated cardiomyopathy, vascular stenosis associated with atherosclerosis, angioplasty treatment, surgical incisions and mechanical trauma.

As used herein “subject in need thereof” refers to a mammal that is suspected of having, or at a risk of having, a disease characterized by vascular smooth muscle cell (“VSMC”) proliferation. Such a mammal can, for example, be a mouse, rat, dog, cat, cow, pig, horse, goat, sheep, rabbit, guinea pig, hamster or primate, such as a human of any gender or age. A mammal is at a risk of having the condition when it, for example, is at an age of being susceptible to the condition, or is genetically predisposed to or has a family history of the condition, or is about to undergo or has recently undergone vascular surgery.

As used herein, the term “treating” includes abrogating, substantially inhibiting, slowing or reversing the progression of a condition, substantially ameliorating clinical or aesthetical symptoms of a condition or substantially preventing the appearance of clinical or aesthetical symptoms of a condition.

According to specific embodiments, the CXCR4-antagonistic peptide of the present invention comprises 4F-benzoyl-TN14003 (SEQ ID NO: 1, also termed herein as BL-8040) or an analog or derivative that is structurally and functionally related to the peptides disclosed in patent applications WO 2002/020561 and WO 2004/020462, also known as “T-140 analogs”, as detailed hereinbelow.

In various particular embodiments, the T-140 analog or derivative has an amino acid sequence as set forth in the following formula (I) or a salt thereof:

(I)
1 2 3 4 5 6 7 8 9 10 11 12 13 14
A₁-A₂-A₃-Cys-Tyr-A₄-A₅-A₆-A₇-A₈-A₉-A₁₀-Cys-A₁₁

wherein:

A₁ is an arginine, lysine, ornithine, citrulline, alanine or glutamic acid residue or a N-a-substituted derivative of these amino acids or A₁ is absent;

A₂ represents an arginine or glutamic acid residue if A₁ is present, or A₂ represents an arginine or glutamic acid residue or a N-α-substituted derivative of these amino acids if A₁ is absent;

A₃ represents an aromatic amino acid residue;

A₄, A₅ and A₉ each independently represents an arginine, lysine, ornithine, citrulline, alanine or glutamic acid residue;

A₆ represents a proline, glycine, ornithine, lysine, alanine, citrulline, arginine or glutamic acid residue;

A₇ represents a proline, glycine, ornithine, lysine, alanine, citrulline or arginine residue;

A₈ represents a tyrosine, phenylalanine, alanine, naphthylalanine, citrulline or glutamic acid residue;

A₉ represents a citrulline, glutamic acid, arginine or lysine residue;

A₁₀ represents an arginine, glutamic acid, lysine or citrulline residue wherein the C-terminal carboxyl may be derivatized;

and the cysteine residue of the 4-position or the 13-position can form a disulfide bond, and the amino acids can be of either L or D form.

Exemplary peptides according to formula (I) are peptides having an amino acid sequence as set forth in any one of SEQ ID NOS:1-72, as presented in Table 1 hereinbelow.

TABLE 1
T-140 and currently preferred T-140 analogs

| | SEQ | |
|---|---|---|
| | ID | |
| Analog | NO: | Amino acid sequence |
| 4F-benzoyl- | 1 | 4F-benzoyl-Arg-Arg-Nal-Cys-Tyr-Cit-Lys-DLys-Pro-Tyr-Arg-Cit-Cys-Arg-NH₂ |
| TN14003 | | |
| AcTC14003 | 2 | Ac-Arg-Arg-Nal-Cys-Tyr-Cit-Lys-DLys-Pro-Tyr-Arg-Cit-Cys-Arg-OH |
| AcTC14005 | 3 | Ac-Arg-Arg-Nal-Cys-Tyr-Arg-Lys-DCit-Pro-Tyr-Arg-Cit-Cys-Arg-OH |
| AcTC14011 | 4 | Ac-Arg-Arg-Nal-Cys-Tyr-Cit-Lys-DCit-Pro-Tyr-Arg-Cit-Cys-Arg-OH |
| AcTC14013 | 5 | Ac-Arg-Arg-Nal-Cys-Tyr-Cit-Lys-DLys-Pro-Tyr-Cit-Cit-Cys-Arg-OH |
| AcTC14015 | 6 | Ac-Cit-Arg-Nal-Cys-Tyr-Cit-Lys-DLys-Pro-Tyr-Arg-Cit-Cys-Arg-OH |
| AcTC14017 | 7 | Ac-Cit-Arg-Nal-Cys-Tyr-Arg-Lys-DCit-Pro-Tyr-Arg-Cit-Cys-Arg-OH |
| AcTC14019 | 8 | Ac-Arg-Arg-Nal-Cys-Tyr-Arg-Lys-DCit-Pro-Tyr-Cit-Cit-Cys-Arg-OH |
| AcTC14021 | 9 | Ac-Cit-Arg-Nal-Cys-Tyr-Arg-Lys-DLys-Pro-Tyr-Cit-Cit-Cys-Arg-OH |
| AcTC14012 | 10 | Ac-Arg-Arg-Nal-Cys-Tyr-Cit-Lys-DCit-Pro-Tyr-Arg-Cit-Cys-Arg-NH₂ |
| AcTC14014 | 11 | Ac-Arg-Arg-Nal-Cys-Tyr-Cit-Lys-DLys-Pro-Tyr-Cit-Cit-Cys-Arg-NH₂ |
| AcTC14016 | 12 | Ac-Cit-Arg-Nal-Cys-Tyr-Cit-Lys-DLys-Pro-Tyr-Arg-Cit-Cys-Arg-NH₂ |
| AcTC14018 | 13 | Ac-Cit-Arg-Nal-Cys-Tyr-Arg-Lys-DCit-Pro-Tyr-Arg-Cit-Cys-Arg-NH₂ |
| AcTC14020 | 14 | Ac-Arg-Arg-Nal-Cys-Tyr-Arg-Lys-DCit-Pro-Tyr-Cit-Cit-Cys-Arg-NH₂ |
| AcTC14022 | 15 | Ac-Cit-Arg-Nal-Cys-Tyr-Arg-Lys-DLys-Pro-Tyr-Cit-Cit-Cys-Arg-NH₂ |
| TE14001 | 16 | H-DGlu-Arg-Nal-Cys-Tyr-Arg-Lys-DLys-Pro-Tyr-Arg-Cit-Cys-Arg-OH |
| TE14002 | 17 | H-Arg-Glu-Nal-Cys-Tyr-Arg-Lys-DLys-Pro-Tyr-Arg-Cit-Cys-Arg-OH |
| TE14003 | 18 | H-Arg-Arg-Nal-Cys-Tyr-Glu-Lys-DLys-Pro-Tyr-Arg-Cit-Cys-Arg-OH |
| TE14004 | 19 | H-Arg-Arg-Nal-Cys-Tyr-Arg-Glu-DLys-Pro-Tyr-Arg-Cit-Cys-Arg-OH |
| TE14005 | 20 | H-Arg-Arg-Nal-Cys-Tyr-Arg-Lys-DGlu-Pro-Tyr-Arg-Cit-Cys-Arg-OH |
| TE14006 | 21 | H-Arg-Arg-Nal-Cys-Tyr-Arg-Lys-DLys-Pro-Tyr-Glu-Cit-Cys-Arg-OH |
| TE14007 | 22 | H-Arg-Arg-Nal-Cys-Tyr-Arg-Lys-DLys-Pro-Tyr-Arg-Cit-Cys-Glu-OH |
| TE14011 | 23 | H-Arg-Arg-Nal-Cys-Tyr-Cit-Lys-DGlu-Pro-Tyr-Arg-Cit-Cys-Arg-NH₂ |
| TE14012 | 24 | H-Arg-Arg-Nal-Cys-Tyr-DGlu-Lys-DCit-Pro-Tyr-Arg-Cit-Cys-Arg-NH₂ |
| TE14013 | 25 | H-Arg-Arg-Nal-Cys-Tyr-DGlu-Lys-DGlu-Pro-Tyr-Arg-Cit-Cys-Arg-NH₂ |
| TE14014 | 26 | H-DGlu-Arg-Nal-Cys-Tyr-Cit-Lys-DGlu-Pro-Tyr-Arg-Cit-Cys-Arg-NH₂ |
| TE14015 | 27 | H-Arg-Arg-Nal-Cys-Tyr-Cit-Lys-DGlu-Pro-DGlu-Arg-Cit-Cys-Arg-NH₂ |
| TE14016 | 28 | H-Arg-Arg-Nal-Cys-Tyr-Cit-Lys-DGlu-Pro-Tyr-Arg-DGlu-Cys-Arg-NH₂ |
| AcTE14014 | 29 | Ac-DGlu-Arg-Nal-Cys-Tyr-Cit-Lys-DGlu-Pro-Tyr-Arg-Cit-Cys-Arg-NH₂ |
| AcTE14015 | 30 | Ac-Arg-Arg-Nal-Cys-Tyr-Cit-Lys-DGlu-Pro-DGlu-Arg-Cit-Cys-Arg-NH₂ |
| AcTE14016 | 31 | Ac-Arg-Arg-Nal-Cys-Tyr-Cit-Lys-DGlu-Pro-Tyr-Arg-DGlu-Cys-Arg-NH₂ |
| TF1: AcTE14011 | 32 | Ac-Arg-Arg-Nal-Cys-Tyr-Cit-Lys-DGlu-Pro-Tyr-Arg-Cit-Cys-Arg-NH₂ |
| TF2: guanyl- | 33 | guanyl-Arg-Arg-Nal-Cys-Tyr-Cit-Lys-DGlu-Pro-Tyr-Arg-Cit-Cys-Arg-NH₂ |
| TE14011 | | |
| TF3: TMguanyl- | 34 | TMguanyl-Arg-Arg-Nal-Cys-Tyr-Cit-Lys-DGlu-Pro-Tyr-Arg-Cit-Cys-Arg-NH₂ |
| TE14011 | | |
| TF4: TMguanyl- | 35 | TMguanyl-Arg-Nal-Cys-Tyr-Cit-Lys-DGlu-Pro-Tyr-Arg-Cit-Cys-Arg-NH₂ |
| TE14011 (2-14) | | |
| TF5: 4F-benzoyl- | 36 | 4F-benzoyl-Arg-Arg-Nal-Cys-Tyr-Cit-Lys-DGlu-Pro-Tyr-Arg-Cit-Cys-Arg-NH₂ |
| TE14011 | | |
| TF6: 2F-benzoyl- | 37 | 2F-benzoyl-Arg-Arg-Nal-Cys-Tyr-Cit-Lys-DGlu-Pro-Tyr-Arg-Cit-Cys-Arg-NH₂ |
| TE14011 | | |
| TF7: APA- | 38 | APA-Arg-Nal-Cys-Tyr-Cit-Lys-DGlu-Pro-Tyr-Arg-Cit-Cys-Arg-NH₂ |
| TE14011 (2-14) | | |
| TF8: desamino-R- | 39 | desamino-R-Arg-Nal-Cys-Tyr-Cit-Lys-DGlu-Pro-Tyr-Arg-Cit-Cys-Arg-NH₂ |
| TE14011 (2-14) | | |
| TF9: guanyl- | 40 | Guanyl-Arg-Nal-Cys-Tyr-Cit-Lys-DGlu-Pro-Tyr-Arg-Cit-Cys-Arg-NH₂ |
| TE14011 (2-14) | | |
| TF10: succinyl- | 41 | succinyl-Arg-Nal-Cys-Tyr-Cit-Lys-DGlu-Pro-Tyr-Arg-Cit-Cys-Arg-NH₂ |
| TE14011 (2-14) | | |
| TF11: glutaryl- | 42 | glutaryl-Arg-Nal-Cys-Tyr-Cit-Lys-DGlu-Pro-Tyr-Arg-Cit-Cys-Arg-NH₂ |
| TE14011 (2-14) | | |
| TF12: | 43 | deaminoTMG-APA-Arg-Nal-Cys-Tyr-Cit-Lys-DGlu-Pro-Tyr-Arg-Cit-Cys-Arg-NH₂ |
| deaminoTMG- | | |
| APA-TE14011 | | |
| (2-14) | | |
| TF15: H-Arg- | 44 | R-CH2-Arg-Nal-Cys-Tyr-Cit-Lys-DGlu-Pro-Tyr-Arg-Cit-Cys-Arg-NH₂ |
| CH2NH- | | |
| RTE14011 (2-14) | | |
| TF17: TE14011 | 45 | H-Arg-Nal-Cys-Tyr-Cit-Lys-DGlu-Pro-Tyr-Arg-Cit-Cys-Arg-NH₂ |
| (2-14) | | |
| TF18: TMguanyl- | 46 | TMguanyl-Arg-Arg-Nal-Cys-Tyr-Cit-Lys-DCit-Pro-Tyr-Arg-Cit-Cys-Arg-NH₂ |
| TC14012 | | |
| TF19: ACA- | 47 | ACA-Arg-Arg-Nal-Cys-Tyr-Cit-Lys-DCit-Pro-Tyr-Arg-Cit-Cys-Arg-NH₂ |
| TC14012 | | |
| TF20: ACA-T140 | 48 | ACA-Arg-Arg-Nal-Cys-Tyr-Arg-Lys-DLys-Pro-Tyr-Arg-Cit-Cys-Arg-OH |
| TZ14011 | 49 | H-Arg-Arg-Nal-Cys-Tyr-Cit-Arg-DLys-Pro-Tyr-Arg-Cit-Cys-Arg-NH₂ |
| AcTZ14011 | 50 | Ac-Arg-Arg-Nal-Cys-Tyr-Cit-Arg-DLys-Pro-Tyr-Arg-Cit-Cys-Arg-NH₂ |
| AcTN14003 | 51 | Ac-Arg-Arg-Nal-Cys-Tyr-Cit-Lys-DLys-Pro-Tyr-Arg-Cit-Cys-Arg-NH₂ |
| AcTN14005 | 52 | Ac-Arg-Arg-Nal-Cys-Tyr-Arg-Lys-DCit-Pro-Tyr-Arg-Cit-Cys-Arg-NH₂ |
| 4F-benzoyl- | 53 | 4F-benzoyl-Arg-Arg-Nal-Cys-Tyr-Cit-Lys-DGlu-Pro-Tyr-Arg-Cit-Cys-Arg-NHMe |
| TN14011-Me | | |
| 4F-benzoyl- | 54 | 4F-benzoyl-Arg-Arg-Nal-Cys-Tyr-Cit-Lys-DGlu-Pro-Tyr-Arg-Cit-Cys-Arg-NHEt |
| TN14011-Et | | |
| 4F-benzoyl- | 55 | 4F-benzoyl-Arg-Arg-Nal-Cys-Tyr-Cit-Lys-DGlu-Pro-Tyr-Arg-Cit-Cys-Arg-NHiPr |
| TN14011-iPr | | |
| 4F-benzoyl- | 56 | 4F-benzoyl-Arg-Arg-Nal-Cys-Tyr-Cit-Lys-DGlu-Pro-Tyr-Arg-Cit-Cys-Arg-tyramine |
| TN14011- | | |
| tyramine | | |
| TA14001 | 57 | H-Ala-Arg-Nal-Cys-Tyr-Arg-Lys-DLys-Pro-Tyr-Arg-Cit-Cys-Arg-OH |
| TA14005 | 58 | H-Arg-Arg-Nal-Cys-Tyr-Ala-Lys-DLys-Pro-Tyr-Arg-Cit-Cys-Arg-OH |
| TA14006 | 59 | H-Arg-Arg-Nal-Cys-Tyr-Arg-Ala-DLys-Pro-Tyr-Arg-Cit-Cys-Arg-OH |
| TA14007 | 60 | H-Arg-Arg-Nal-Cys-Tyr-Arg-Lys-DAla-Pro-Tyr-Arg-Cit-Cys-Arg-OH |
| TA14008 | 61 | H-Arg-Arg-Nal-Cys-Tyr-Arg-Lys-DLys-Ala-Tyr-Arg-Cit-Cys-Arg-OH |
| TA14009 | 62 | H-Arg-Arg-Nal-Cys-Tyr-Arg-Lys-DLys-Pro-Ala-Arg-Cit-Cys-Arg-OH |
| TA14010 | 63 | H-Arg-Arg-Nal-Cys-Tyr-Arg-Lys-DLys-Pro-Tyr-Ala-Cit-Cys-Arg-OH |
| TC14001 | 64 | H-Cit-Arg-Nal-Cys-Tyr-Arg-Lys-DLys-Pro-Tyr-Arg-Cit-Cys-Arg-OH |
| TC14003 | 65 | H-Arg-Arg-Nal-Cys-Tyr-Cit-Lys-DLys-Pro-Tyr-Arg-Cit-Cys-Arg-OH |
| TN14003 | 66 | H-Arg-Arg-Nal-Cys-Tyr-Cit-Lys-DLys-Pro-Tyr-Arg-Cit-Cys-Arg-NH₂ |
| TC14004 | 67 | H-Arg-Arg-Nal-Cys-Tyr-Arg-Cit-DLys-Pro-Tyr-Arg-Cit-Cys-Arg-OH |
| TC14012 | 68 | H-Arg-Arg-Nal-Cys-Tyr-Cit-Lys-DCit-Pro-Tyr-Arg-Cit-Cys-Arg-NH₂ |
| T-140 | 69 | H-Arg-Arg-Nal-Cys-Tyr-Arg-Lys-DLys-Pro-Tyr-Arg-Cit-Cys-Arg-OH |
| TC14011 | 70 | H-Arg-Arg-Nal-Cys-Tyr-Cit-Lys-DCit-Pro-Tyr-Arg-Cit-Cys-Arg-OH |
| TC14005 | 71 | H-Arg-Arg-Nal-Cys-Tyr-Arg-Lys-DCit-Pro-Tyr-Arg-Cit-Cys-Arg-OH |
| TC14018 | 72 | H-Cit-Arg-Nal-Cys-Tyr-Arg-Lys-DCit-Pro-Tyr-Arg-Cit-Cys-Arg-NH₂ |

According to a specific embodiment, in each one of SEQ ID NOS:1-72, two cysteine residues are coupled in a disulfide bond.

In another embodiment, the analog or derivative has an amino acid sequence as set forth in SEQ ID NO:65 (H-Arg-Arg-Nal-Cys-Tyr-Cit-Lys-DLys-Pro-Tyr-Arg-Cit-Cys-Arg-OH; TC14003).

In another embodiment, the peptide used in the compositions and methods of the invention consists essentially of an amino acid sequence as set forth in SEQ ID NO:1. In another embodiment, the peptide used in the compositions and methods of the invention comprises an amino acid sequence as set forth in SEQ ID NO:1. In another embodiment, the peptide is at least 60%, at least 70% or at least 80% homologous to SEQ ID NO:1. In another embodiment, the peptide is at least 90% homologous to SEQ ID NO:1. In another embodiment, the peptide is at least about 95% homologous to SEQ ID NO:1. Each possibility represents a separate embodiment of the present invention.

In various other embodiments, the peptide is selected from SEQ ID NOS:1-72, wherein each possibility represents a separate embodiment of the present invention.

In another embodiment, the peptide has an amino acid sequence as set forth in any one of SEQ ID NOS: 1-4, 10, 46, 47, 51-56, 65, 66, 68, 70 and 71. In another embodiment, the peptide has an amino acid sequence as set forth in any one of SEQ ID NOS: 4, 10, 46, 47, 68 and 70. In another embodiment, the peptide has an amino acid sequence as set forth in any one of SEQ ID NOS:1, 2, 51, 65 and 66. In another embodiment, the peptide has an amino acid sequence as set forth in any one of SEQ ID NOS:53-56.

In an embodiment, the peptide has an amino acid sequence as set forth in SEQ ID NO:1. In another embodiment, the peptide has an amino acid sequence as set forth in SEQ ID NO:2. In another embodiment, the peptide has an amino acid sequence as set forth in SEQ ID NO:51. In another embodiment, the peptide has an amino acid sequence as set forth in SEQ ID NO:66.

According to a preferred embodiment, the CXCR4 antagonist is as set forth in SEQ ID NO: 1.

The CXCR4 antagonist and possibly other active ingredients (e.g., for the treatment of vascular smooth muscle cell proliferation diseases as described herein) can each be administered to the subject as active ingredients per se, or in a pharmaceutical composition(s) where (each of) the active ingredients is mixed with suitable carriers or excipients.

Thus, for example, in the case of restenosis, the antagonistic peptide can be administered in conjunction with other therapeutics found effective to limit or eliminate restenosis, such as, for example, anti-platelet, anti-coagulation, anti-inflammatory, and vasodilation therapeutics.

As used, herein a “pharmaceutical composition” refers to a preparation of one or more of the active ingredients described herein with other chemical components such as physiologically suitable carriers and excipients. The purpose of a pharmaceutical composition is to facilitate administration of a compound to an organism.

Herein the term “active ingredient” refers to the peptides accountable for the biological effect. Optionally, a plurality of active ingredient may be included in the formulation, as further described hereinbelow.

Hereinafter, the phrases “physiologically acceptable carrier” and “pharmaceutically acceptable carrier”, which may be used interchangeably, refer to a carrier or a diluent that does not cause significant irritation to an organism and does not abrogate the biological activity and properties of the administered compound.

Herein, the term “excipient” refers to an inert substance added to a pharmaceutical composition to further facilitate administration of an active ingredient. Examples, without limitation, of excipients include calcium carbonate, calcium phosphate, various sugars and types of starch, cellulose derivatives, gelatin, vegetable oils, and polyethylene glycols.

Techniques for formulation and administration of drugs may be found in the latest edition of “Remington's Pharmaceutical Sciences”, Mack Publishing Co., Easton, Pa., which is herein fully incorporated by reference (Remington: The Science and Practice of Pharmacy, Gennaro, A., Lippincott, Williams & Wilkins, Philadelphia, Pa., 20^{th} ed, 2000).

Pharmaceutical compositions of the present invention may be manufactured by processes well known in the art, e.g., by means of conventional mixing, dissolving, granulating, dragee-making, levigating, emulsifying, encapsulating, entrapping, or lyophilizing processes.

Pharmaceutical compositions for use in accordance with the present invention thus may be formulated in conventional manner using one or more physiologically acceptable carriers comprising excipients and auxiliaries, which facilitate processing of the active ingredients into preparations that can be used pharmaceutically. Proper formulation is dependent upon the route of administration chosen.

The pharmaceutical composition may be administered in a number of ways depending on whether local or systemic treatment is desired, and on the area to be treated. Administration may be accomplished by methods known to those skilled in the art such as intravenously or by use of a catheter or a stent to direct treatment to an affected area. Intravascular devices and their use are known to those skilled in the art.

For example, local administration to the relevant traumatized vessel by way of a catheter is one form of delivery. The peptide (and possibly other active agents, such as described hereinabove) is administered in the vicinity of the lesion via a catheter from inside the lumen, e.g., a porous balloon as described by Wolinsky and Thung, JACC 15: 475-481 (1990), or through the adventitia (i.e., the most outer layer of the vessel wall) with materials aiding slow release of the active ingredient. Other slow release techniques for local delivery include coating stents with the active ingredient compound, e.g., using a binder or gel described in Wilensky et al., Trends in Cardiovascular Med. 3:163-170 (1993). According to a specific embodiment, intracoronary administration using a catheter is contemplated.

Systemic administration is contemplated according to some embodiments of the invention.

The amount of a composition to be administered will, of course, be dependent on the subject being treated, the severity of the affliction, the manner of administration, the judgment of the prescribing physician, etc.

According to a specific embodiment the therapeutically effective amount comprises a single administration.

In one embodiment, the CXCR4 antagonist of the invention or the pharmaceutical composition comprising same is administered subcutaneously.

In one embodiment, the CXCR4 antagonist of the invention or the pharmaceutical composition comprising same is administered orally.

For injection, the active ingredients of the pharmaceutical composition may be formulated in aqueous solutions (e.g., WFI), preferably in physiologically compatible buffers such as Hank's solution, Ringer's solution, or physiological salt buffer.

Pharmaceutical compositions for potential administration include aqueous solutions of the active preparation in water-soluble form. Additionally, suspensions of the active ingredients may be prepared as appropriate oily or water-based injection suspensions. Suitable lipophilic solvents or vehicles include fatty oils such as sesame oil, or synthetic fatty acid esters such as ethyl oleate, triglycerides, or liposomes. Aqueous injection suspensions may contain substances that increase the viscosity of the suspension, such as sodium carboxymethyl cellulose, sorbitol, or dextran. Optionally, the suspension may also contain suitable stabilizers or agents that increase the solubility of the active ingredients, to allow for the preparation of highly concentrated solutions.

Alternatively, the active ingredient may be in powder form for constitution with a suitable vehicle, e.g., a sterile, pyrogen-free, water-based solution, before use.

Alternative embodiments include depots providing sustained release or prolonged duration of activity of the active ingredient in the subject, as are well known in the art.

Pharmaceutical compositions suitable for use in the context of the present invention include compositions wherein the active ingredients are contained in an amount effective to achieve the intended purpose. Determination of a therapeutically effective amount is well within the capability of those skilled in the art, especially in light of the detailed disclosure provided herein.

For any preparation used in the methods of the invention, the therapeutically effective amount or dose can be estimated initially from in vitro and cell culture assays. For example, a dose can be formulated in animal models to achieve a desired concentration or titer. Such information can be used to more accurately determine useful doses in humans.

Toxicity and therapeutic efficacy of the active ingredients described herein can be determined by standard pharmaceutical procedures in vitro, in cell cultures or experimental animals (see the Examples section which follows, and Sekido et al. 2002 Cancer Genet Cytogenet 137(1):33-42). The data obtained from these in vitro and cell culture assays and animal studies can be used in formulating a range of dosage for use in human. The dosage may vary depending upon the dosage form employed and the route of administration utilized. The exact formulation, route of administration and dosage can be chosen by the individual physician in view of the patient's condition. (See e.g., Fingl, et al., 1975, in “The Pharmacological Basis of Therapeutics”, Ch. 1 p. 1).

According to a specific embodiment, treatment with the peptide is accompanied by other Gold standard treatments contemplated for the contemplated diseases. The selection of the specific treatment depends on the specific type of the disease.

Exemplary treatments for cardiac diseases include, but are not limited to, Ace inhibitors, anticoagulation drugs, Beta blockers, Statins.

A more detailed list can be found at:

Specific examples include, but are not limited to:

### Angiotensin-Converting Enzyme (ACE) Inhibitors

Commonly prescribed include:

### Captopril (Capoten)

### Enalapril (Vasotec)

### Fosinopril (Monopril)

### Lisinopril (Prinivil, Zestril)

### Perindopril (Aceon)

### Quinapril (Accupril)

### Ramipril (Altace)

### Trandolapril (Mavik)

### Angiotensin II Receptor Blockers (or Inhibitors)

(Also known as ARBs or Angiotensin-2 Receptor Antagonists)
Commonly prescribed include:

### Candesartan (Atacand)

### Losartan (Cozaar)

### Valsartan (Diovan)

### Angiotensin-Receptor Neprilysin Inhibitors (ARNIs)

ARNIs are a new drug combination of a neprilysin inhibitor and an ARB.

### Sacubitril/valsartan

### I_{f} Channel Blocker (or Inhibitor)

This drug class reduces the heart rate, similar to another class of drugs called beta blockers.

### Ivabradine (Corlanor)

### Beta Blockers

(Also known as Beta-Adrenergic Blocking Agents)
Commonly prescribed include:

### Bisoprolol (Zebeta)

Metoprolol succinate (Toprol XL)

### Carvedilol (Coreg)

### Carvedilol CR (Coreg CR) Toprol XL

### Aldosterone Antagonists

Commonly prescribed include:

### Spironolactone (Aldactone)

### Eplerenone (Inspra)

Hydralazine and Isosorbide Dinitrate (Specifically Benefits African Americans with Heart Failure)
Commonly prescribed:
Hydralazine and isosorbide dinitrate (combination drug)—(Bidil)

### Diuretics

(Also known as Water Pills)
Commonly prescribed include:

### Furosemide (Lasix)

### Bumetanide (Bumex)

### Torsemide (Demadex)

### Chlorothiazide (Diuril)

### Amiloride (Midamor Chlorthalidone (Hygroton)

### Hydrochlorothiazide or HCTZ (Esidrix, Hydrodiuril)

### Indapamide (Lozol)

### Metolazone (Zaroxolyn)

### Triamterene (Dyrenium)

As used herein the term “about” refers to ±10%

The terms “comprises”, “comprising”, “includes”, “including”, “having” and their conjugates mean “including but not limited to”.

The term “consisting of” means “including and limited to”.

The term “consisting essentially of” means that the composition, method or structure may include additional ingredients, steps and/or parts, but only if the additional ingredients, steps and/or parts do not materially alter the basic and novel characteristics of the claimed composition, method or structure.

As used herein, the singular form “a”, “an” and “the” include plural references unless the context clearly dictates otherwise. For example, the term “a compound” or “at least one compound” may include a plurality of compounds, including mixtures thereof.

Throughout this application, various embodiments of this invention may be presented in a range format. It should be understood that the description in range format is merely for convenience and brevity and should not be construed as an inflexible limitation on the scope of the invention. Accordingly, the description of a range should be considered to have specifically disclosed all the possible subranges as well as individual numerical values within that range. For example, description of a range such as from 1 to 6 should be considered to have specifically disclosed subranges such as from 1 to 3, from 1 to 4, from 1 to 5, from 2 to 4, from 2 to 6, from 3 to 6 etc., as well as individual numbers within that range, for example, 1, 2, 3, 4, 5, and 6. This applies regardless of the breadth of the range.

Whenever a numerical range is indicated herein, it is meant to include any cited numeral (fractional or integral) within the indicated range. The phrases “ranging/ranges between” a first indicate number and a second indicate number and “ranging/ranges from” a first indicate number “to” a second indicate number are used herein interchangeably and are meant to include the first and second indicated numbers and all the fractional and integral numerals therebetween.

As used herein the term “method” refers to manners, means, techniques and procedures for accomplishing a given task including, but not limited to, those manners, means, techniques and procedures either known to, or readily developed from known manners, means, techniques and procedures by practitioners of the chemical, pharmacological, biological, biochemical and medical arts.

As used herein, the term “treating” includes abrogating, substantially inhibiting, slowing or reversing the progression of a condition, substantially ameliorating clinical or aesthetical symptoms of a condition or substantially preventing the appearance of clinical or aesthetical symptoms of a condition.

The antagonistic peptide as described herein can be combined with other treatment modalities. These other treatments include medication (e.g., blood pressure medication, calcium channel blockers, digitalis, anti-arrhythmics, ACE inhibitors, anti-coagulants, immunosuppressants, pain relievers, vasodilators, etc.), angioplasty, stent placement, coronary artery bypass graft, cardiac assist device (e.g., left ventricular assist device, balloon pump), pacemaker placement, heart transplantation, etc.

It is appreciated that certain features of the invention, which are, for clarity, described in the context of separate embodiments, may also be provided in combination in a single embodiment. Conversely, various features of the invention, which are, for brevity, described in the context of a single embodiment, may also be provided separately or in any suitable subcombination or as suitable in any other described embodiment of the invention. Certain features described in the context of various embodiments are not to be considered essential features of those embodiments, unless the embodiment is inoperative without those elements.

Various embodiments and aspects of the present invention as delineated hereinabove and as claimed in the claims section below find experimental support in the following examples.

### EXAMPLES

Reference is now made to the following examples, which together with the above descriptions, illustrate the invention in a non-limiting fashion.

Generally, the nomenclature used herein and the laboratory procedures utilized in the present invention include molecular, biochemical, microbiological and recombinant DNA techniques. Such techniques are thoroughly explained in the literature. See, for example, “Molecular Cloning: A laboratory Manual” Sambrook et al., (1989); “Current Protocols in Molecular Biology” Volumes I-III Ausubel, R. M., Ed. (1994); Ausubel et al., “Current Protocols in Molecular Biology”, John Wiley and Sons, Baltimore, Md. (1989); Perbal, “A Practical Guide to Molecular Cloning”, John Wiley & Sons, New York (1988); Watson et al., “Recombinant DNA”, Scientific American Books, New York; Birren et al. (Eds.) “Genome Analysis: A Laboratory Manual Series”, Vols. 1-4, Cold Spring Harbor Laboratory Press, New York (1998); methodologies as set forth in U.S. Pat. Nos. 4,666,828; 4,683,202; 4,801,531; 5,192,659 and 5,272,057; “Cell Biology: A Laboratory Handbook”, Volumes I-III Cellis, J. E., Ed. (1994); “Culture of Animal Cells—A Manual of Basic Technique” by Freshney, Wiley-Liss, N. Y. (1994), Third Edition; “Current Protocols in Immunology” Volumes I-III Coligan J. E., Ed. (1994); Stites et al. (Eds.), “Basic and Clinical Immunology” (8th Edition), Appleton & Lange, Norwalk, Conn. (1994); Mishell and Shiigi (Eds.), “Selected Methods in Cellular Immunology”, W. H. Freeman and Co., New York (1980); available immunoassays are extensively described in the patent and scientific literature, see, for example, U.S. Pat. Nos. 3,791,932; 3,839,153; 3,850,752; 3,850,578; 3,853,987; 3,867,517; 3,879,262; 3,901,654; 3,935,074; 3,984,533; 3,996,345; 4,034,074; 4,098,876; 4,879,219; 5,011,771 and 5,281,521; “Oligonucleotide Synthesis” Gait, M. J., Ed. (1984); “Nucleic Acid Hybridization” Hames, B. D., and Higgins S. J., Eds. (1985); “Transcription and Translation” Hames, B. D., and Higgins S. J., Eds. (1984); “Animal Cell Culture” Freshney, R. I., Ed. (1986); “Immobilized Cells and Enzymes” IRL Press, (1986); “A Practical Guide to Molecular Cloning” Perbal, B., (1984) and “Methods in Enzymology” Vol. 1-317, Academic Press; “PCR Protocols: A Guide To Methods And Applications”, Academic Press, San Diego, Calif. (1990); Marshak et al., “Strategies for Protein Purification and Characterization—A Laboratory Course Manual” CSHL Press (1996); all of which are incorporated by reference as if fully set forth herein. Other general references are provided throughout this document. The procedures therein are believed to be well known in the art and are provided for the convenience of the reader. All the information contained therein is incorporated herein by reference.

### Materials and Methods

Cell Lines

Human NB cell lines SK-N-BE(2), Shy-SY5Y and MHH-NB-11; prostate cancer cell line PC3; multiple myeloma cell line RPMI-8266. The SK-N-BE(2) cell line was kindly provided by Prof. Elizabeth Beierle (Birmingham Ala., USA). The human SHY-SY5Y and MHH-NB-11 cell lines were kindly provided by Prof. Isaac Witz (Tel Aviv University, Israel). All the other cell lines were purchased from ATCC. Cells were maintained at log growth in RPMI medium (Biological Industries) supplemented with 10% fetal calf serum (FCS), 1 mM L-glutamine, 100 U/ml penicillin, and 0.01 mg/ml streptomycin (Biological Industries) in a humidified atmosphere of 5% CO₂ at 37° C.

Tumor Tissue Array

Tissue analysis of CXCR4 expression in neuroblastoma (NB) was analyzed in a tissue microarray (US BioMax MC809). Analysis was determined by scoring the staining intensity by two independent investigators.

Flow Cytometry Analysis

Cells were stained with human specific directly-labeled antibodies and analyzed by FACScalibur (Becton Dickinson Immunocytometry Systems), using the CellQuest software. CXCR4 monoclonal antibody, clone 12G5 (R&D Systems). BCL-2 monoclonal antibody, clone BCL-2/100 (eBioscience). For internal staining, cells were first fixed with 4% paraformaldehyde, permeabilized with 0.1% saponin.

Cell Proliferation Assay

Cells were seeded at 2×10⁴ cells/1 ml per well into a 24-well plate in medium supplemented with 0.1% FCS with or without various concentrations of CXCL12 (PeproTech EC). On days 2, 4 and 7, cells were harvested, stained with propidium iodide (PI, Sigma), and the number of viable cells was determined using FACS analysis.

Soft Agar Colony Assay

An agar base layer was prepared as follows: 45 mL RPMI plus 12% FCS was mixed with 15 mL RPMI 32 plus 12% FCS and 15 mL 2.5% agar in double-distilled water. The tumor cells were suspended in RPMI plus 10% FCS. Cell suspension was mixed in a ratio of 1:3 with the agar base solution. This mixture was then plated on top of a preformed solid agar base. CXCL12 (100 ng/mL) or BL-8040 (20 μM) or anti-CXCR4 (1 μg/ml) were added to the mixture. Fourteen days later, the number of colonies was counted in 10 different fields.

Cell Survival Assay

A total of 1×10⁵ cells/mL were cultured in 24-well plates with 1000 mL RPMI 1640 medium supplemented with 10% FCS. 24 hours later, the medium was changed to RPMI 1640 supplemented with either 1% FCS, with BL-8040 or AMD3100 or ABT199. Then 24 hours later, the cells were harvested, washed, and stained with PI (Sigma). The number of live cells was counted and analyzed using FACS analysis.

For SK-N-BE(2)-CXCR4 overexpression, 1×10⁴ cells were seeded onto a 96-well microtiter xCELLigence assay plate (E-Plate) (ACEA Biosciences Inc.) and placed on the Real-Time xCELLigence Cell Analyzer (Roche Applied Science) platform at 37° C. to measure the cell index every 5 min.

Protein Extraction

SK-N-BE(2) cells were seeded into a 12-well plate at 2×10⁵/1 ml of medium per well. The cells were incubated for 48 hours and supernatants were collected. For whole cells: tumor samples and cells were lysed by the addition of lysis buffer containing 50 mM Tris-HCl PH 7.6, 150 mM NaCl, 5 mM EDTA pH 8, 0.5% NP40 and protease inhibitor cocktail (Roche Diagnostics). Lysate was incubated with buffer for 15-20 minutes and then centrifuges at 14,000 rpm, 15 min, at 4° C. Protein amounts were determined by Bradford assay (Bio-Rad).

ELISA Assay

CXCL12 protein levels of cell supernatants or tumor lysates were determined using sandwich-type ELISA commercially available kit according to the manufacture's protocol (R&D Systems). The absorbance was read at 450 nm.

RNA Isolation

Total RNA from various cell lines and mouse tissues was isolated using Trizol reagent (Invitrogen) according to the manufacture's protocol, followed by DNaseI treatment using the DNaseI Kit where needed (Ambion). The concentrations were measured by Nanodrop (ND spectrophotometer) and the integrity was analyzed by gel electrophoreses on 1% agarose gel.

Quantitative Real Time RT-PCR

cDNA was synthesized from 0.5-1 μg total RNA using the Quanta Biosciences qScript™ cDNA Synthesis Kit (95047-100) for mRNA analysis, and using the qScript™ microRNA cDNA Synthesis Kit (95107-100) for miRNAs analysis. Quantiative PCR (qPCR) of miRNAs and mRNA was performed using the CFX384, C1000 touch thermal cycler (Bio-Rad) and a SYBR Green PCR Kit: Quanta Cat. #84018 and #84071, respectively. The fold expression and statistical significance were calculated using the 2-ΔΔ Ct method. All experiments were performed in triplicate.

Primers
All primers were purchased from IDT-syntezza.
CXCR4;
s
(SEQ ID NO: 73)
GAACCCTGTTTCCGTGAAGA;
as
(SEQ ID NO: 74)
CTTGTCCGTCATGCTTCTCA.
CXCL12;
s
(SEQ ID NO: 75)
GTCTGTTGTTGTTCTTCAGCC;
as
(SEQ ID NO: 76)
ATGCCCATGCCGATTCTTCG.
BCL-2;
s
(SEQ ID NO: 77)
GATAACGGAGGCTGGGATGC;
as
(SEQ ID NO: 78)
TCACTTGTGGCCCAGATAGG.
CCND1;
s
(SEQ ID NO: 79)
TTGCCCTCTGTGCCACAGAT;
as
(SEQ ID NO: 80)
TCAGGTTCAGGCCTTGCACT.
miR-15a
(SEQ ID NO: 81)
TAGCAGCACATAATGGTTTGTG.
miR-16-1
(SEQ ID NO: 82)
TAGCAGCACGTAAATATTGGCG.
HPRT;
s
(SEQ ID NO: 83)
GGACAGGACTGAACGTCTTGC;
as
(SEQ ID NO: 84)
CAACACTTCGTGGGGTCCTT.
c-myc;
s
(SEQ ID NO: 85)
GGGGCTTTATCTAACTCGCTGTA;
as
(SEQ ID NO: 86)
TATGGGCAAAGTTTCGTGGAT.

Western Blot Analysis

Protein extracts were equally loaded onto 10% SDS-polyacrylamide gel, electrophorized and transferred onto a polyvinylidenedifluoride (PVDF) membrane (Bio-Rad Laboratories). Then, membranes were blocked and incubated with primary specific antibody O.N. at 4° C. For BCL-2 mouse monoclonal (Abcam). For cycD1 rabbit monoclonal (Thermo). For pERK1/2 rabbit monoclonal (Cell signaling). For β-Actin mouse monoclonal (MP biomedicals). After the washing procedure (3 times, 10 min in washing solution), the membrane was incubated with a secondary immunopure HRP-conjugated antibody, anti-mouse or anti rabbit (Envision; Dako) (1/10,000), washed (5 times, 6 min) and detected with the EZ-ECL kit (Biological Industries). Photon emission was identified by ChemiDoc MP imaging system (Bio-Rad). Intensities of protein bands were quantified by computerized densitometry using Image lab software (Bio-Rad).

Immunohistochemistry (IHC)

For histological analysis, tumor tissue was cut into 5-mm sections, deparaffinized with xylene, and hydrated through graded ethanol. Endogenous peroxidase was blocked by incubation for 5 minutes in 3% H₂O₂. A 25-mM citrate buffer (pH 6.0) was used for antigen retrieval, cooked in a pressure cooker for 20 minutes, and left to cool for 30 minutes at room temperature. Slides were washed in Optimax (Pharmatrade) and incubated with primary Ab diluted in CAS-Block (Zymed Laboratories). For Ki67 1:100 (Thermo Scientific), for CXCR4 anti-human CXCR4 monoclonal antibody clone 12G5 1:100 (R&D), for CXCL12 monoclonal CXCL12 antibody 79018 (R&D), for BCL-2 monoclonal BCL-2 antibody clone 124 (cell marque) and for cyclinD1 monoclonal BCL-1 antibody clone sp4 (spring). Staining overnight at 4° C. For all stainings, a conjugated horseradish peroxidase secondary Ab anti mouse or anti rabbit (Envision; Dako) was used for 30 minutes and developing was done with diaminobenzidine for 5 minutes followed by counterstaining with hematoxylin. For ki67, BCL-2 and Cyclin D1, staining levels were quantified using the Image Pro Analyzer image analysis software.

Transduction of Cell Lines

In order to stably overexpress CXCR4, SK-N-BE(2) cells were transduced with the lentiviral bicistronic vector harboring a CXCR4 expression cassette. This was done using a three-plasmid system: pHR′-CMVCXCR4-IRES-GFP-WPRE; envelope coding plasmid VSV-G and a packaging construct CMVDR8.91 according to previously published protocol (23). For transduction of SK-N-BE(2) cells, 1 ml of viral supernatant was used, which is equivalent to an m.o.i. of ˜9.

Flow cytometric analysis (FACS) analyzed the percentage CXCR4+ cells. PC3-CXCR4 and RPMI-CXCR4 cells were previously generated (6,24).

Transfections

Transfections were performed using Lipofectamine 2000 transfection reagent (Invitrogen) according to the manufacturer's protocol. Plasmids were purchased from Dharmacon. MiR-15a: miRIDIAN microRNA hsa-miR-15a-5p mimic. MiR-16-1: miRIDIAN microRNA hsa-miR-16-5p mimic. Negative control miR: miRIDIAN microRNA inhibitor negative control. AntagomiR-15a: miRIDIAN microRNA hsa-miR-15a-5p hairpin inhibitor. AntagomiR-16-1: miRIDIAN microRNA hsa-miR-16-5p hairpin inhibitor. AntagomiR negative control: miRIDIAN microRNA hairpin inhibitor negative control 1. All miRNA-mimics and antagomiRs were transfected at 50 nM or 100 nM. To determine the efficiency of transfection, at 48 hours post-transfection, the cells were analyzed for RNA.

Gene Expression Array

RNA was isolated from tumors and subjected to gene expression profiling using GeneChip human Gene 1.0 ST Array (Affymetrix). The gene expression values were extracted using the Partek Genomics Suite 6.6 software and, after thresholding and filtering procedures, were submitted to fold change and cluster analyses (Gene Expression Omnibus [GEO] GSE94426).

In-Vivo Orthotopic NB Model

Human SK-N-BE(2) cells were orthotopically injected into the left adrenal gland of 6-8 weeks old NSG mice. A 27-gauge needle was introduced through the left adrenal fat pad and 5×10∧5 cells/20 μl PBS were inoculated to the adrenal gland. NSG mice were maintained under defined flora conditions at the Hebrew University Pathogen-Free Animal Facility. Tumor growth and volume were monitored bi-weekly by using T2-weighted MRI until tumors reached the ethical limit volume and mice were randomized to drug-treated or control mice. Mice in the treated group were subjected to 2 treatment protocols. In the first treatment protocol BL-8040 (400 μg) was injected daily, starting three days following inoculation, and was continued for 35 days. In the second treatment protocol mice were monitored for 21 days. As of day 7, mice were treated with a daily injection of BL-8040 (400 μg) for 14 consecutive days.

Study Approval

All animal experiments were approved by the Animal Care Committee of the Hebrew University.

MR Imaging

Magnetic resonance imaging (MRI) scans were performed on a horizontal 4.7T Biospec spectrometer (Bruker Medical) with a 3.5-cm birdcage coil. Mice were anesthetized with Isoflurane (Nicholas Piramal, India; 2% in a mixture of 30:70 O2:N2O). Tumor volume was assessed bi-weekly using T2-weighted (T2W) fast spin echo images (repetition time=2,000 ms; echo time=37 ms; in plane resolution=117 μm; slice thickness-1 mm). Tumor volume was manually assessed using Analyze-7.0 (BIR). For each subject, an exponential growth curve was fitted to the tumor volume data points (Matlab software).

CXCR4 and BCL-2 Inhibitors

BL-8040 (BKT-140, 4F-benzoyl-TN14003) was kindly provided by BioLineRx Ltd. AMD3100 was purchased from Sigma-Aldrich. ABT199 was purchased from Selleck Chemicals.

Statistics

Data are presented as means±SD or ±SE. Statistical comparison of means was performed by a two-tailed unpaired Student's t test. Differences with a P<0.05 were determined as statistically significant. Statistical analysis of NB tumor mice survival following early treatment was performed using the Mantel-cox test.

### Example 1

### CXCR4 Expression in Human NB Tumors and Tumor-Derived Cell Lines

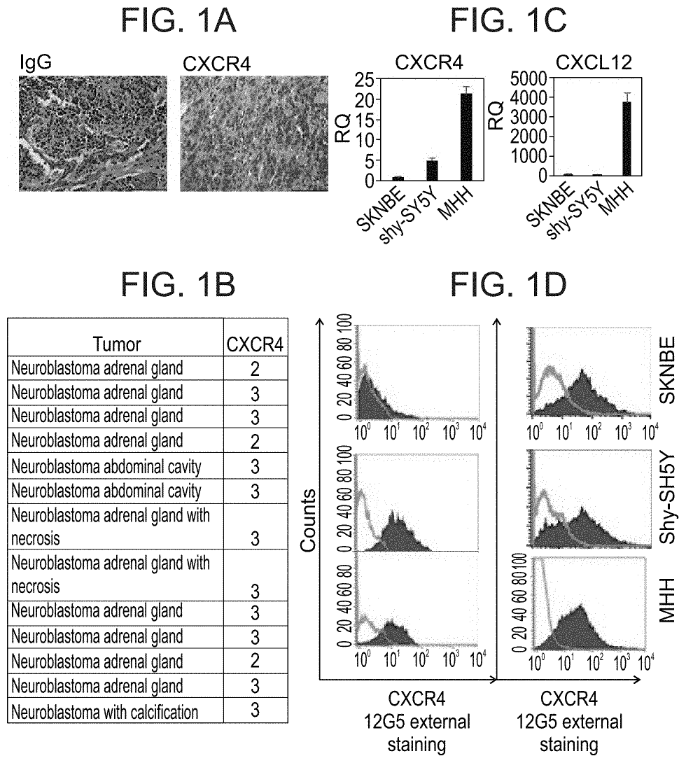

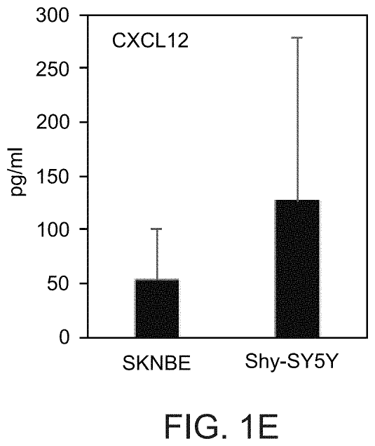

To appreciate the significance of CXCR4 to neuroblastoma (NB) development, CXCR4 expression was measured in a tissue array composed of 13 NB samples and several cell lines derived from NB tumors. All sections examined stained strongly for CXCR4 (FIGS. 1A, 1B). All cell lines tested expressed CXCR4 at variable levels measured by qPCR (FIG. 1C). CXCL12 expression was detected using qPCR (FIG. 1C) and ELISA (FIG. 1E). To determine CXCR4 protein levels, FACS staining for CXCR4 was performed in the three cell lines. Membrane staining with the 12G5 antibody revealed CXCR4 staining only on the Shy-SY5Y and MHH-NB-11 cell lines; however, intracellular staining showed that the SK-N-BE(2) cell line expressed CXCR4 as well (FIG. 1D). The results confirm, as previously published(10), that CXCR4 is markedly expressed in NB tumors. In addition, these results confirm the presence of CXCR4 in all cell lines tested. Out of the three cell lines, cells expressing the highest (Shy-SY5Y) and lowest (SK-N-BE(2)) CXCR4 levels were used for further analysis.

### Example 2

### CXCR4 Effects Proliferation and Viability of Neuroblastoma Cell Lines In-Vitro

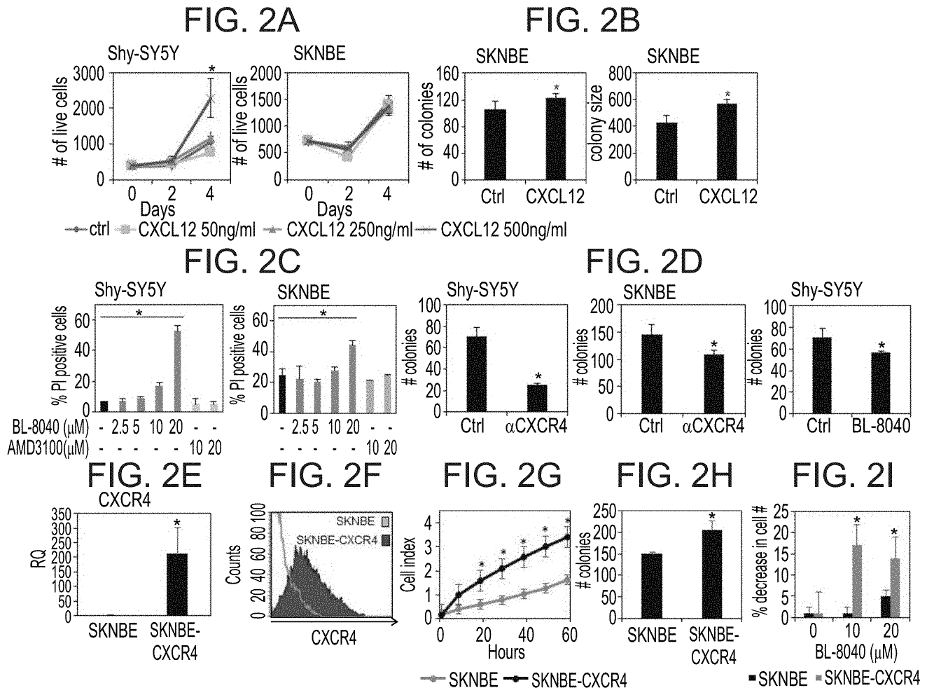

The effect of CXCR4 on the growth of NB cells was determined. To examine whether CXCR4 has a proliferative effect on the Shy-SY5Y and SK-N-BE(2) cell lines, cells were incubated with different doses of the ligand CXCL12. Upon incubation with CXCL12 (500 ng/ml), the CXCR4 high expressing cell line Shy-SY5Y displayed increased proliferation (FIG. 2A). On the other hand, the low CXCR4 expressing cell line SK-N-BE(2) displayed no response to CXCL12 (FIG. 2A). However, SK-N-BE(2) cells demonstrated a high colony formation ability when incubated with CXCL12 (100 ng/ml) (FIG. 2B), indicating that in both NB cell lines CXCR4 has a role in cell proliferation.

Next, the present inventor investigated whether blocking CXCR4 using the antagonists BL-8040 or AMD3100 would influence NB cell viability. Both cell lines tested displayed decreased viability while exposed to increasing concentrations of BL-8040 (FIG. 2C) but not to AMD3100. In addition, blocking of CXCR4 with either BL-8040 or antibodies against CXCR4 reduces colony formation (FIG. 2D).

To further investigate the influence of CXCR4 on the proliferation of NB cells, SK-N-BE(2)-CXCR4 cells were generated. The level of CXCR4 in the overexpressing cells was considerably higher than in control cells as determined by qPCR (FIG. 2E) and flow cytometry analysis (FIG. 2F). SK-N-BE(2)-CXCR4 cells demonstrated higher growth rate as well as increased colony formation ability in comparison to the native cell line (FIGS. 2G, 2H). The CXCR4 overexpressing cell line also displayed increased sensitivity to BL-8040 treatment in-vitro (FIG. 2I). Overall, these results demonstrate that CXCR4 has a functional effect on NB cell line proliferation and viability and that expression levels of CXCR4 determine its dependency on the receptor.

### Example 3

### CXCR4 Inhibits Neuroblastoma Tumor Growth In-Vivo

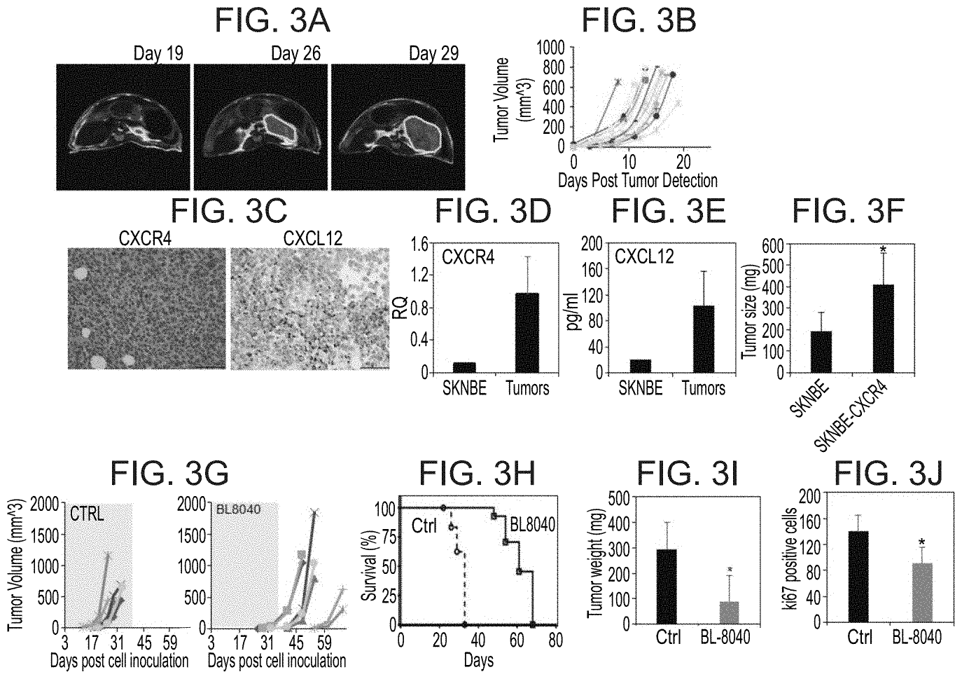

To investigate the role of CXCR4 on NB tumors in-vivo, an orthotopic xenograft tumor model was established. The SK-N-BE(2) cells were chosen for this model since they displayed a high inoculation rate. SK-N-BE(2) cells were injected to the left adrenal gland and mice were monitored bi-weekly for 65 days using MRI, presenting with an exponential growth pattern (FIG. 3B). The anatomic location of tumors was confined to the adrenal gland in accordance with the human tumors (FIG. 3A). In tumor sections, expression of CXCR4 and CXCL12 were abundantly detected by IHC (FIG. 3C). Accordingly, evaluation of CXCR4 mRNA levels in tumors revealed a 10-fold upregulation of the receptor compared to the SK-N-BE(2) cells grown in-vitro (FIG. 3D). Notable levels of CXCL12 were also detected in tumors by ELISA (FIG. 3E).

To further test the influence of CXCR4 presence on tumor growth in-vivo SK-N-BE(2) cells overexpressing CXCR4 were implanted to the left adrenal of mice. Tumors arising from SK-N-BE(2) cells overexpressing CXCR4 were 2-folds larger than the tumors from the native cell line 21 days post inoculation (FIG. 3F).

Following the establishment of the xenograft model the influence of BL-8040 on tumor development in-vivo was assessed. As long as BL-8040 was administered, tumor growth attenuation was exhibited. Discontinuation of BL-8040 treatment, resulted in tumors surge in an exponential growth pattern similar to controls (FIG. 3G). Survival of animals following this treatment regimen increased significantly from an average of 35 days to 61 days (FIG. 3H).

Next, it was determined whether administering BL-8040 post tumor formation would also influence tumor development in mice. Tumors extracted from the BL-8040 treated mice were comparably smaller as measured by a 3-fold reduction in tumor weight (FIG. 3I). In addition, compared to controls, treated mice showed reduced Ki67 staining, indicating a lower proliferative state (FIG. 3J).

These results indicate that SK-N-BE(2) cells in-vivo express CXCR4 as well as CXCL12. Moreover, as reported previously, CXCR4 overexpression enhances tumor growth. Most significantly, it was demonstrated that treatment with the CXCR4 inhibitor BL-8040, is capable of impairing NB tumor formation and growth resulting in increased survival of animals.

### Example 4

### Identification of miR-15a/16-1 as Target microRNA's Regulated by CXCR4 Inhibitor BL-8040

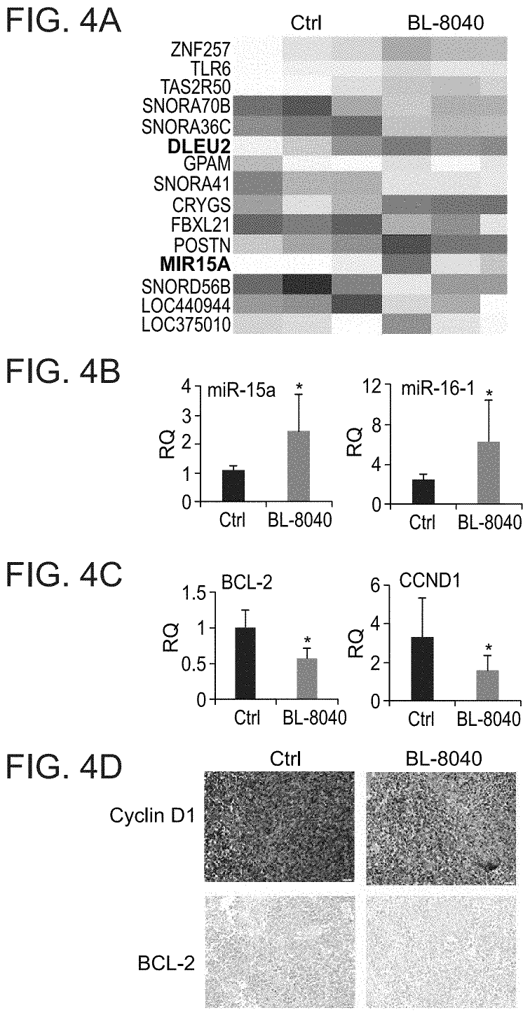

In order to identify genes that are important for NB development and are regulated by CXCR4, the expression patterns of genes between BL-8040 treated and un-treated NB tumors was compared. Gene expression was assessed by screening a human DNA chip array, using the Affymatrix gene chip expression analysis system. Among the differentially expressed genes, increased expression of miR-15a and its parent gene DLEU2 was found (FIG. 4A). Further validation of these results was obtained by analyzing RNA samples from nine treated and un-treated tumors. MiR-15a and a second miR located on the same locus miR-16-1 were both significantly elevated in treated tumors (FIG. 4B). Focusing on two of their target genes: BCL-2 and CCND1, it was found that the expression levels of BCL-2 and CCND1 mRNA in treated versus un-treated tumors was significantly downregulated (FIG. 4C). Additionally, treated tumors sections compared to controls sections showed a significant reduction in Cyclin D1 expression (56%). BCL-2 staining was weak and therefore the difference between treated and a control was not significant (FIG. 4D, 36%).

### Example 5

### BL-8040 Affects Survival of NB Cells In-Vitro by Upregulating miR-15a/16-1

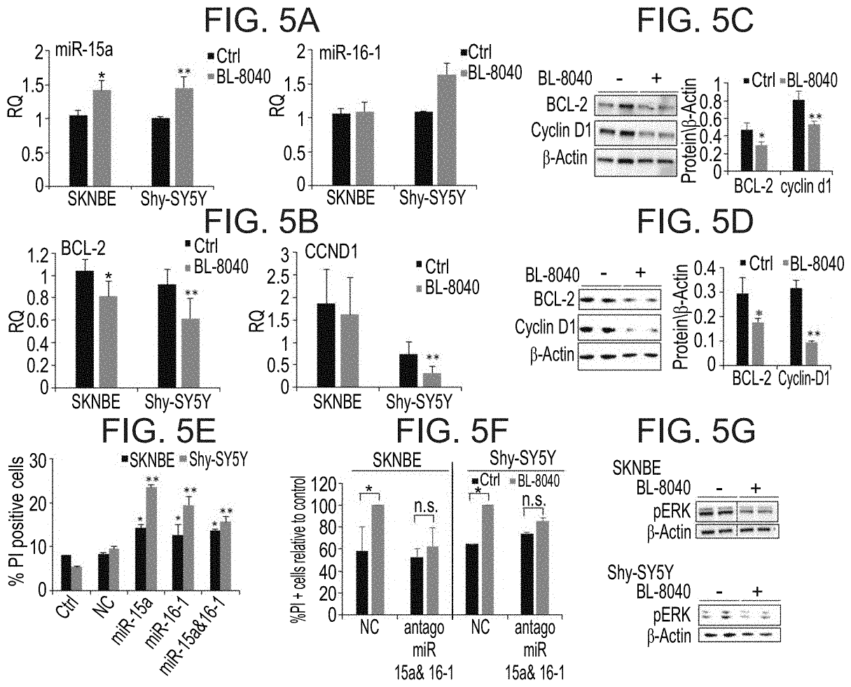

In an effort to extend our previous results, the influence of the CXCR4 inhibitor BL-8040 on the miR-15a/16-1 axis in-vitro was assessed. SK-N-BE(2) and Shy-SY5Y cells were incubated with BL-8040 (20 μM) and RNA was extracted for microRNA evaluation. QPCR revealed a significant increase in miR-15a, but not miR-16-1 in both cells treated with BL-8040 (FIG. 5A). Consequently, the mRNA of BCL-2 was reduced in both cells and the mRNA of CCND1 was reduced only in the Shy-SY5Y cells (FIG. 5B). Nonetheless, western blot analysis of protein extracts from both cells incubated with BL-8040, showed a reduction in both BCL-2 and cycD1 (FIGS. 5C-D). Additionally, BL-8040 decreased ERK phosphorylation in both cells as shown by western blot (FIG. 5G).

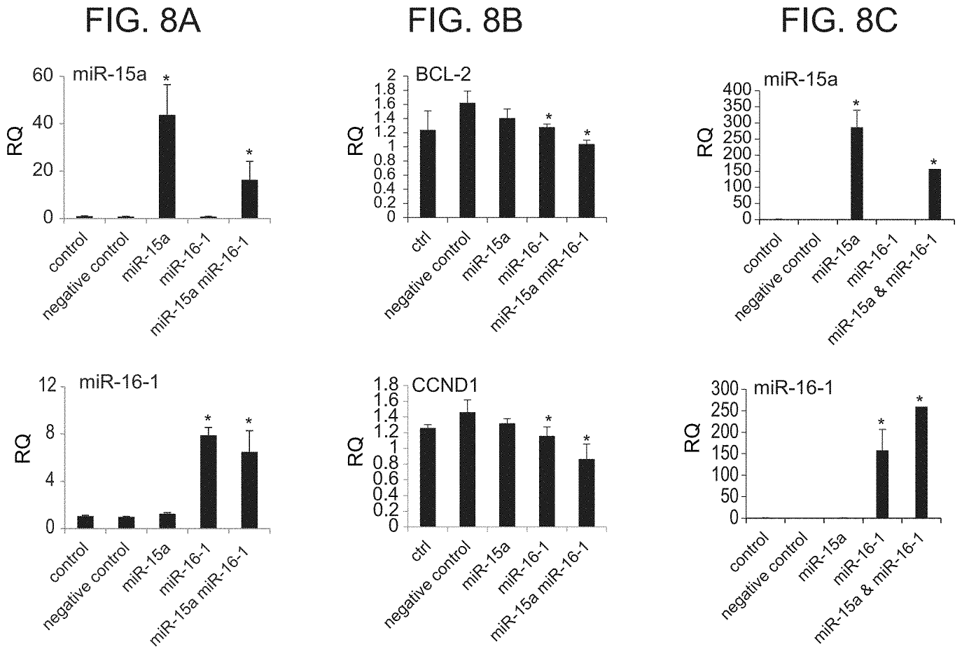

Cell death can be achieved by introducing miR-15a and/or miR-16-1 exogenously into tumor cells (25,26). To test whether miR-15a/16-1 could regulate cell death in NB cells, miR-15a and/or miR-16-1 were transfected into SK-N-BE(2) and Shy-SY5Y cells. Results are shown in FIGS. 8A-C. The levels of the corresponding miR increased between 7 and 286-folds following transfection. Accordingly, reduction in the level of both target genes occurred following transfection with miR-16-1 or both miRs. Elevated levels of miR-15a/16-1 were found to directly induce cell death in SK-N-BE(2) and Shy-SY5Y cells as demonstrated by higher PI incorporation compared to control and negative control miR transfected cells (FIG. 5E). These data indicate that when the level of miR-15a/16-1 are increased in the NB cell lines tested, genes important for cell survival are downregulated resulting in cell death.

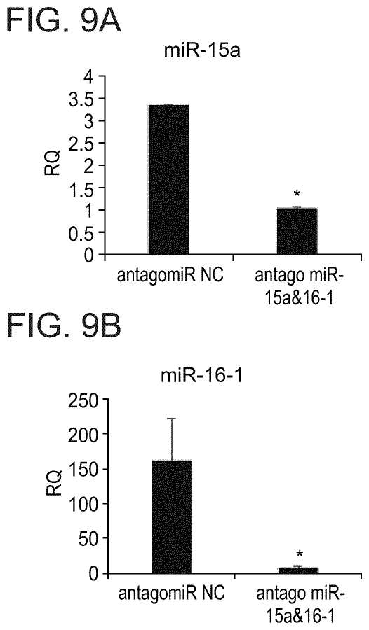

To further explore the involvement of miR-15a/16-1 in BL-8040 induced killing of NB cells, miR-15a/16-1 endogenous cellular levels were reduced by transfection with antagomiRs. Transfection efficacy was evaluated by quantifying miR-15a/16-1 RNA levels by qPCR (FIGS. 9A-B). Following antagomiR transfection, cells were treated with BL-8040 for 48 hours and viability was analyzed. SK-N-BE(2) and Shy-SY5Y cells transfected with antago miR-15a and antago miR-16-1 displayed reduced cell death when treated with BL-8040 (FIG. 5F). These results indicate that BL-8040-induced cell death is dependent on miR-15a/16-1 in both NB cells.

### Example 6

### The CXCR4 Receptor Upregulates miR-15a/16-1 Downregulating BCL-2 and CCND1

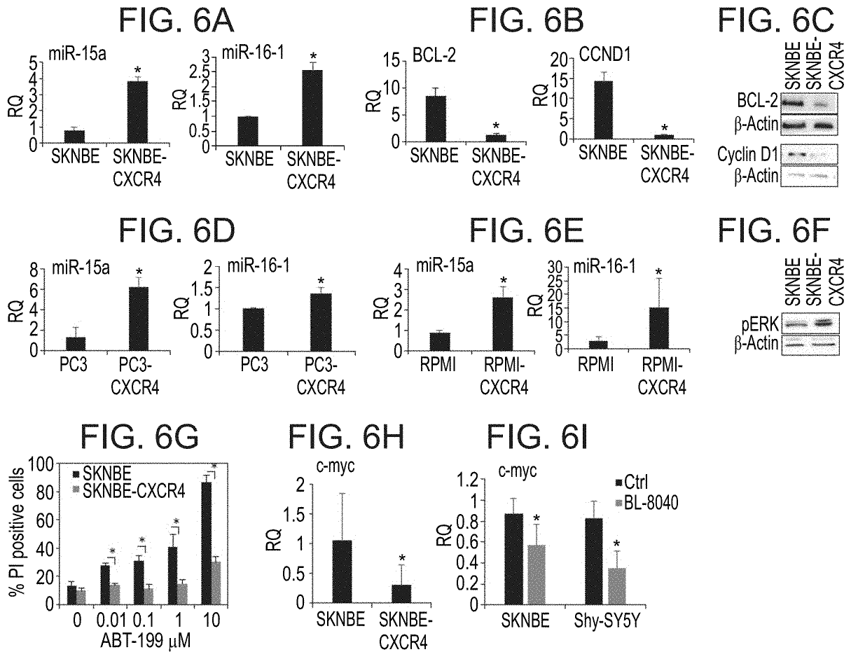

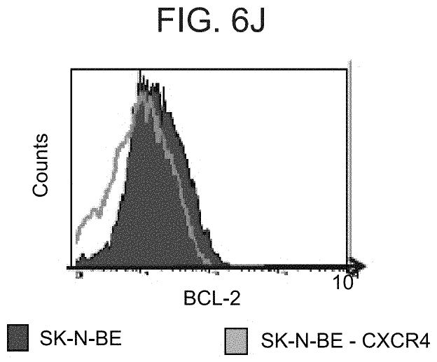

To further study the role of CXCR4 in miR-15a/16-1 regulation their expression pattern in SK-N-BE(2)-CXCR4 cells was tested. Surprisingly, miR-15a/16-1, as measured by qPCR, were significantly upregulated in the SK-N-BE(2)-CXCR4 cells (FIG. 6A). Furthermore, BCL-2 mRNA was downregulated by 4-folds and CCND1 was downregulated by 14-folds in the SK-N-BE(2)-CXCR4 cells (FIG. 6B). This decrease resulted also in a reduction in the protein levels of BCL-2 and CycD1 tested by western blot (FIG. 6C) and flow cytometry (FIG. 6J). Remarkably, when SK-N-BE(2)-CXCR4 cells were incubated with increased concentrations of the BCL-2 inhibitor ABT-199 they displayed high resistance to the inhibitor compared to the native cell line (FIG. 6G). This finding supports the result that BCL-2 levels are downregulated in the CXCR4 overexpressing cells.

To better understand the mechanism by which CXCR4 upregulates miR-15a and miR-16-1 in NB cells. The expression pattern of PAX5, e2f1, e2f7 and c-myc all transcription factors known to target the DLEU2 promoter ((27-29) were examined in the NB cells treated with BL-8040 and in the SK-N-BE(2)-CXCR4 cells. c-myc, a negative regulator of the DLEU2 gene, was reduced significantly both in the NB cells treated with BL-8040 and in the SK-N-BE(2)-CXCR4 cells (FIGS. 6H-I). These results unveil an additional levels of regulation of miR-15a and miR-16-1 controlled by CXCR4.

To elaborate on the connection between CXCR4 activation and miR-15a/16-1 expression, this expression pattern was measured in other tumor types. Two additional cell lines were examined: the multiple myeloma RPMI8266 cell line and the prostate cancer PC3 cell line. The levels of miR-15a/16-1 in RPMI-CXCR4 and PC3-CXCR4 cells were significantly upregulated compared to the native controls (FIGS. 6D-E).

It has previously been demonstrated that both PC3-CXCR4 (6) and RPMI-CXCR4 (24) cells exhibit an increase in ERK1/2 phosphorylation induced by CXCR4 overexpression. Respectively, an increase in ERK1/2 phosphorylation was detected in the SK-N-BE(2) cells overexpressing CXCR4 (FIG. 6F). These results demonstrate that the MAPK pathway is induced upon CXCR4 activation. Taken together with the previous data, it is suggested that in CXCR4 overexpressing cells, miR-15a/16-1 are upregulated, inducing BCL-2 and CCND1 downregulation, whereas signaling pathways such as the MAPK are activated thus supporting cell proliferation and tumor progression.

Although the invention has been described in conjunction with specific embodiments thereof, it is evident that many alternatives, modifications and variations will be apparent to those skilled in the art. Accordingly, it is intended to embrace all such alternatives, modifications and variations that fall within the spirit and broad scope of the appended claims.

All publications, patents and patent applications mentioned in this specification are herein incorporated in their entirety by reference into the specification, to the same extent as if each individual publication, patent or patent application was specifically and individually indicated to be incorporated herein by reference. In addition, citation or identification of any reference in this application shall not be construed as an admission that such reference is available as prior art to the present invention. To the extent that section headings are used, they should not be construed as necessarily limiting.

### REFERENCES

### Other References are Cited Throughout the Application

1. De Bernardi B, Nicolas B, Boni L, Indolfi P, Carli M, Cordero Di Montezemolo L, et al. Disseminated neuroblastoma in children older than one year at diagnosis: comparable results with three consecutive high-dose protocols adopted by the Italian Co-Operative Group for Neuroblastoma. J Clin Oncol Off J Am Soc Clin Oncol. 2003 Apr. 15; 21(8):1592-601.
2. Berthold F, Boos J, Burdach S, Erttmann R, Henze G, Hermann J, et al. Myeloablative megatherapy with autologous stem-cell rescue versus oral maintenance chemotherapy as consolidation treatment in patients with high-risk neuroblastoma: a randomised controlled trial. Lancet Oncol. 2005 September; 6(9):649-58.
3. Chatterjee S, Behnam Azad B, Nimmagadda S. The intricate role of CXCR4 in cancer. Adv Cancer Res. 2014; 124:31-82.
4. Barbero S, Bonavia R, Bajetto A, Porcile C, Pirani P, Ravetti J L, et al. Stromal cell-derived factor 1alpha stimulates human glioblastoma cell growth through the activation of both extracellular signal-regulated kinases 1/2 and Akt. Cancer Res. 2003 Apr. 15; 63(8):1969-74.
5. Smith M C P, Luker K E, Garbow J R, Prior J L, Jackson E, Piwnica-Worms D, et al. CXCR4 regulates growth of both primary and metastatic breast cancer. Cancer Res. 2004 Dec. 1; 64(23):8604-12.
6. Darash-Yahana M, Pikarsky E, Abramovitch R, Zeira E, Pal B, Karplus R, et al. Role of high expression levels of CXCR4 in tumor growth, vascularization, and metastasis. FASEB J Off Publ Fed Am Soc Exp Biol. 2004 August; 18(11):1240-2.
7. Porcile C, Bajetto A, Barbieri F, Barbero S, Bonavia R, Biglieri M, et al. Stromal cell-derived factor-1alpha (SDF-1alpha/CXCL12) stimulates ovarian cancer cell growth through the EGF receptor transactivation. Exp Cell Res. 2005 Aug. 15; 308(2):241-53.
8. Fahham D, Weiss I D, Abraham M, Beider K, Hanna W, Shlomai Z, et al. In vitro and in vivo therapeutic efficacy of CXCR4 antagonist BKT140 against human non-small cell lung cancer. J Thorac Cardiovasc Surg. 2012 November; 144(5):1167-1175.e1.
9. Heinrich E L, Lee W, Lu J, Lowy A M, Kim J. Chemokine CXCL12 activates dual CXCR4 and CXCR7-mediated signaling pathways in pancreatic cancer cells. J Transl Med. 2012; 10:68.
10. Russell H V, Hicks J, Okcu M F, Nuchtern J G. CXCR4 expression in neuroblastoma primary tumors is associated with clinical presentation of bone and bone marrow metastases. J Pediatr Surg. 2004 October; 39(10):1506-11.
11. Geminder H, Sagi-Assif O, Goldberg L, Meshel T, Rechavi G, Witz I P, et al. A possible role for CXCR4 and its ligand, the CXC chemokine stromal cell-derived factor-1, in the development of bone marrow metastases in neuroblastoma. J Immunol Baltim Md 1950. 2001 Oct. 15; 167(8):4747-57.
12. Zhang L, Yeger H, Das B, Irwin M S, Baruchel S. Tissue microenvironment modulates CXCR4 expression and tumor metastasis in neuroblastoma. Neoplasia N Y N. 2007 January; 9(1):36-46.
13. Meier R, Mühlethaler-Mottet A, Flahaut M, Coulon A, Fusco C, Louache F, et al. The chemokine receptor CXCR4 strongly promotes neuroblastoma primary tumour and metastatic growth, but not invasion. PloS One. 2007; 2(10):e1016.
14. Jensen T, Vadasz S, Phoenix K, Claffey K, Parikh N, Finck C. Descriptive analysis of tumor cells with stem like phenotypes in metastatic and benign adrenal tumors. J Pediatr Surg. 2015 September; 50(9):1493-501.
15. Peled A, Wald O, Burger J. Development of novel CXCR4-based therapeutics. Expert Opin Investig Drugs. 2012 March; 21(3):341-53.
16. Beider K, Begin M, Abraham M, Wald H, Weiss ID, Wald O, et al. CXCR4 antagonist 4F-benzoyl-TN14003 inhibits leukemia and multiple myeloma tumor growth. Exp Hematol. 2011 March; 39(3):282-92.
17. Beider K, Darash-Yahana M, Blaier O, Koren-Michowitz M, Abraham M, Wald H, et al. Combination of imatinib with CXCR4 antagonist BKT140 overcomes the protective effect of stroma and targets CML in vitro and in vivo. Mol Cancer Ther. 2014 May; 13(5):1155-69.
18. Tamamura H, Hori A, Kanzaki N, Hiramatsu K, Mizumoto M, Nakashima H, et al. T140 analogs as CXCR4 antagonists identified as anti-metastatic agents in the treatment of breast cancer. FEBS Lett. 2003 Aug. 28; 550(1-3):79-83.
19. Cimmino A, Cabin G A, Fabbri M, Iorio M V, Ferracin M, Shimizu M, et al. miR-15 and miR-16 induce apoptosis by targeting BCL2. Proc Natl Acad Sci USA. 2005 Sep. 27; 102(39):13944-9.
20. Deshpande A, Pastore A, Deshpande A J, Zimmermann Y, Hutter G, Weinkauf M, et al. 3′UTR mediated regulation of the cyclin D1 proto-oncogene. Cell Cycle Georget Tex. 2009 Nov. 1; 8(21):3592-600.
21. Bonci D, Coppola V, Musumeci M, Addario A, Giuffrida R, Memeo L, et al. The miR-15a-miR-16-1 cluster controls prostate cancer by targeting multiple oncogenic activities. Nat Med. 2008 November; 14(11):1271-7.
22. Pekarsky Y, Croce C M. Role of miR-15/16 in CLL. Cell Death Differ. 2015 January; 22(1):6-11.
23. Gropp M, Reubinoff B. Lentiviral vector-mediated gene delivery into human embryonic stem cells. Methods Enzymol. 2006; 420:64-81.
24. Beider K, Rosenberg E, Bitner H, Shimoni A, Leiba M, Koren-Michowitz M, et al. The Sphingosine 1 Phosphate Modulator FTY720 Targets Multiple Myeloma via the CXCR4/CXCL12 Pathway. Clin Cancer Res Off J Am Assoc Cancer Res. 2016 Oct. 3;
25. Bonci D, Coppola V, Musumeci M, Addario A, Giuffrida R, Memeo L, et al. The miR-15a-miR-16-1 cluster controls prostate cancer by targeting multiple oncogenic activities. Nat Med. 2008 November; 14(11):1271-7.
26. Cimmino A, Calin G A, Fabbri M, Iorio M V, Ferracin M, Shimizu M, et al. miR-15 and miR-16 induce apoptosis by targeting BCL2. Proc Natl Acad Sci. 2005 Sep. 27; 102(39):13944-9.
27. Chung E Y, Dews M, Cozma D, Yu D, Wentzel E A, Chang T-C, et al. c-Myb oncoprotein is an essential target of the dleu2 tumor suppressor microRNA cluster. Cancer Biol Ther. 2008 November; 7(11):1758-64.
28. Chu J, Zhu Y, Liu Y, Sun L, Lv X, Wu Y, et al. E2F7 overexpression leads to tamoxifen resistance in breast cancer cells by competing with E2F1 at miR-15a/16 promoter. Oncotarget. 2015 Oct. 13; 6(31):31944-57.
29. Xue G, Yan H-L, Zhang Y, Hao L-Q, Zhu X-T, Mei Q, et al. c-Myc-mediated repression of miR-15-16 in hypoxia is induced by increased HIF-2α and promotes tumor angiogenesis and metastasis by upregulating FGF2. Oncogene. 2015 Mar. 12; 34(11):1393-406.
30. Rubin J B, Kung A L, Klein R S, Chan J A, Sun Y, Schmidt K, et al. A small-molecule antagonist of CXCR4 inhibits intracranial growth of primary brain tumors. Proc Natl Acad Sci USA. 2003 Nov. 11; 100(23):13513-8.
31. Chen X, Zhu Y, Han L, Lu H, Hao X, Dong Q. Chemokine receptor 4 gene silencing blocks neuroblastoma metastasis in vitro. Neural Regen Res. 2014 May 15; 9(10):1063-7.
32. De Clercq E. The bicyclam AMD3100 story. Nat Rev Drug Discov. 2003 July; 2(7):581-7.
33. Reynolds C P. Detection and treatment of minimal residual disease in high-risk neuroblastoma. Pediatr Transplant. 2004 June; 8 Suppl 5:56-66.
34. Domanska U M, Kruizinga R C, Nagengast W B, Timmer-Bosscha H, Huls G, de Vries E G E, et al. A review on CXCR4/CXCL12 axis in oncology: no place to hide. Eur J Cancer Oxf Engl 1990. 2013 January; 49(1):219-30.
35. Casimiro M C, Velasco-Velazquez M, Aguirre-Alvarado C, Pestell R G. Overview of cyclins D1 function in cancer and the CDK inhibitor landscape: past and present. Expert Opin Investig Drugs. 2014 March; 23(3):295-304.
36. Croce C M, Reed J C. Finally, An Apoptosis-Targeting Therapeutic for Cancer. Cancer Res. 2016 Oct. 15; 76(20):5914-20.
37. Cantley L C. The phosphoinositide 3-kinase pathway. Science. 2002 May 31; 296(5573): 1655-7.
38. Mo W, Chen J, Patel A, Zhang L, Chau V, Li Y, et al. CXCR4/CXCL12 mediate autocrine cell-cycle progression in NF1-associated malignant peripheral nerve sheath tumors. Cell. 2013 February 28; 152(5):1077-90.
39. Klein U, Lia M, Crespo M, Siegel R, Shen Q, Mo T, et al. The DLEU2/miR-15a/16-1 Cluster Controls B Cell Proliferation and Its Deletion Leads to Chronic Lymphocytic Leukemia. Cancer Cell. 2010 January; 17(1):28-40.
40. Parker H, Rose-Zerilli M J J, Parker A, Chaplin T, Wade R, Gardiner A, et al. 13q deletion anatomy and disease progression in patients with chronic lymphocytic leukemia. Leukemia. 2011 March; 25(3):489-97.
41. Xue G, Yan H-L, Zhang Y, Hao L-Q, Zhu X-T, Mei Q, et al. c-Myc-mediated repression of miR-15-16 in hypoxia is induced by increased HIF-2α and promotes tumor angiogenesis and metastasis by upregulating FGF2. Oncogene. 2015 Mar. 12; 34(11):1393-406.
42. Scala S, Ottaiano A, Ascierto P A, Cavalli M, Simeone E, Giuliano P, et al. Expression of CXCR4 predicts poor prognosis in patients with malignant melanoma. Clin Cancer Res Off J Am Assoc Cancer Res. 2005 Mar. 1; 11(5):1835-41.
43. Jiang Y-P, Wu X-H, Shi B, Wu W-X, Yin G-R. Expression of chemokine CXCL12 and its receptor CXCR4 in human epithelial ovarian cancer: an independent prognostic factor for tumor progression. Gynecol Oncol. 2006 October; 103(1):226-33.
44. Burger J A, Stewart D J, Wald O, Peled A. Potential of CXCR4 antagonists for the treatment of metastatic lung cancer. Expert Rev Anticancer Ther. 2011 April; 11(4):621-30.
45. Liberman J, Sartelet H, Flahaut M, Mühlethaler-Mottet A, Coulon A, Nyalendo C, et al. Involvement of the CXCR7/CXCR4/CXCL12 axis in the malignant progression of human neuroblastoma. PloS One. 2012; 7(8):e43665.
46. Kremer K N, Peterson K L, Schneider P A, Meng X W, Dai H, Hess A D, et al. CXCR4 chemokine receptor signaling induces apoptosis in acute myeloid leukemia cells via regulation of the Bcl-2 family members Bcl-XL, Noxa, and Bak. J Biol Chem. 2013 Aug. 9; 288(32):22899-914.
47. Colamussi M L, Secchiero P, Gonelli A, Marchisio M, Zauli G, Capitani S. Stromal derived factor-1alpha (SDF-1 alpha) induces CD4+ T cell apoptosis via the functional up-regulation of the Fas (CD95)/Fas ligand (CD95L) pathway. J Leukoc Biol. 2001 February; 69(2):263-70.
48. Drury L J, Wendt M K, Dwinell M B. CXCL12 chemokine expression and secretion regulates colorectal carcinoma cell anoikis through Bim-mediated intrinsic apoptosis. PloS One. 2010; 5(9):e12895.
49. Goldsmith Z G, Dhanasekaran D N. G protein regulation of MAPK networks. Oncogene. 2007 May 14; 26(22):3122-42.

1. A method of treating a disease associated with vascular smooth muscle cell proliferation in a subject in need thereof, with the proviso that the disease is not cancer, the method comprising administering to the subject a therapeutically effective amount of a CXCR4-antagonistic peptide comprising SEQ ID NO: 1, thereby treating the disease.

2. (canceled)

3. A device for cardiac delivery comprising a CXCR4-antagonistic peptide comprising SEQ ID NO. 1.

4. The method of claim 1, wherein said disease is a cardiac disease.

5. The method of claim 4, wherein said cardiac disease is selected from the group consisting of cardiac hypertrophy, transplant arteriosclerosis, atherosclerosis, angioplasty restenosis, ventricular stenosis or cardiac vein bypass stenosis.

6. The method of claim 1, wherein said disease is selected from the group consisting of essential hypertension, thrombosis, stenosis, ventricular stenosis, diabetic macroangiopathy, myocardial infarction, stroke, vascular dementia, intimal hyperplasia, restenosis, angioplasty restenosis, cardiac vein bypass stenosis, transplant arteriosclerosis and atherosclerosis.

7. The method of claim 1, wherein said administering comprises local administering.

8. (canceled)

9. The device of claim 3, being a stent or a catheter.
